(19) United States
(12) Patent Application Publication
(10) Pub. No.: US 2015/0171339 A1
(43) Pub. Date: Jun. 18, 2015

(54) MATERIAL FOR ORGANIC ELECTROLUMINESCENCE DEVICE AND ORGANIC ELECTROLUMINESCENCE DEVICE USING THE SAME

(71) Applicant: SAMSUNG DISPLAY CO., LTD., Yongin-City (KR)

(72) Inventors: Naoya SAKAMOTO, Yokohama (JP); Yasuo MIYATA, Yokohama (JP)

(21) Appl. No.: 14/572,436

(22) Filed: Dec. 16, 2014

(30) Foreign Application Priority Data
Dec. 18, 2013 (JP) 2013-261648

(51) Int. Cl. H01L 51/00; H01L 51/50; C07D 305/14; C07D 407/12; C07D 495/04; C07F 7/08

(52) U.S. Cl. CPC H01L 51/0067; C07D 495/04; H01L 51/0071; C07F 7/0812; H01L 51/0094; H01L 51/0072; H01L 51/0073; C07D 305/14; C07D 407/12; H01L 51/5012

(57) ABSTRACT

A material for an organic electroluminescence (EL) device includes a triarylamine derivative represented by the following Formula 1:

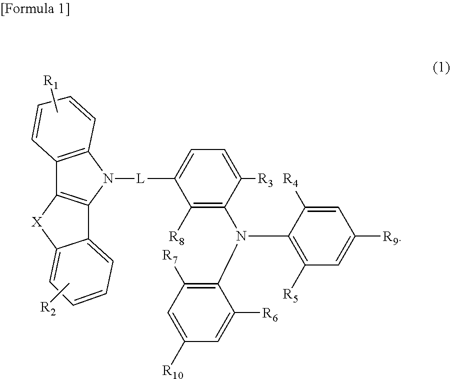

### CROSS-REFERENCE TO RELATED APPLICATIONS

Japanese Patent Application No. 2013-261648, filed on Dec. 18, 2013, in the Japanese Intellectual Property Office, and entitled: “Material for Organic Electroluminescence Device and Organic Electroluminescence Device Using the Same,” is incorporated by reference herein in its entirety.

### BACKGROUND

1. Field

Embodiments relate to a material for an organic electroluminescence device and an organic electroluminescence device including the same.

2. Description of the Related Art

Organic electroluminescence (EL) displays are one type of image displays that have been actively developed. Unlike a liquid crystal display and the like, the organic EL display is so-called a self-luminescent display that recombines holes and electrons injected from a positive electrode and a negative electrode in an emission layer to thus emit light from a light-emitting material including an organic compound of the emission layer, thereby performing display.

### SUMMARY

Embodiments are directed to A material for an organic electroluminescence (EL) device, the material including a triarylamine derivative represented by the following Formula 1:

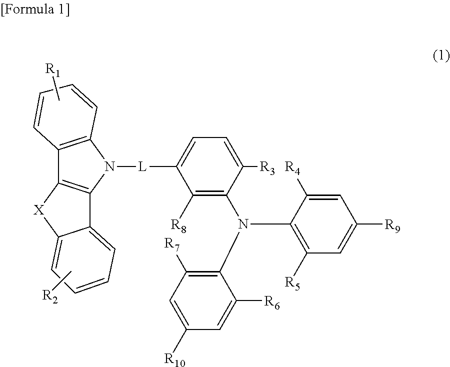

In Formula 1:

X may be an oxygen atom or a sulfur atom,

R₁ and R₂ may independently be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom,

R₃ to R₁₀ may independently be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, oxygen, sulfur, or a bond to another one of R₃ to R₁₀, and

L may be an arylene group.

Two or more of R₃ to R₈ in Formula 1 may be combined to each other to form a saturated or unsaturated ring.

R₃ and R₄ in Formula 1 may be combined to each other to form a chemical structure represented by the following Formula 2:

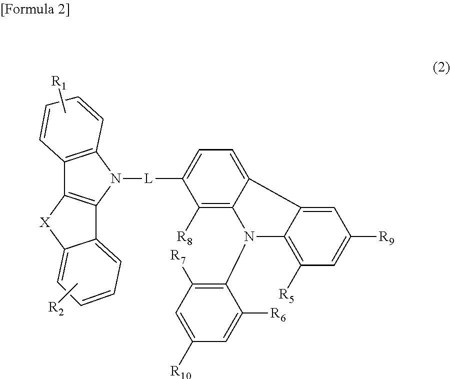

In Formula 2:

X may be an oxygen atom or a sulfur atom,

R₁ and R₂ may independently be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom,

R₅ to R₁₀ may independently be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom, and

L may be an arylene group.

Two or more of R₅ to R₈ in Formula 1 may be combined to each other to form a saturated or unsaturated ring.

Embodiments are also directed to an organic electroluminescence (EL) device including a material for an organic EL device including a triarylamine derivative represented by the following Formula 3, the triarylamine derivative represented by Formula 3 being in at least one layer of an emission layer and a stacking layer disposed between an emission layer and a positive electrode:

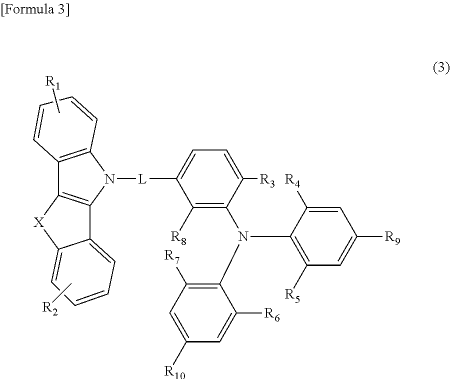

In Formula 3:

X may be an oxygen atom or a sulfur atom,

R₁ and R₂ may independently be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom,

R₃ to R₁₀ may independently be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, oxygen, sulfur, or a bond to another one of R₃ to R₁₀, and

L may be an arylene group.

Two or more of R₃ to R₈ in Formula 3 may be combined to each other to form a saturated or unsaturated ring.

R₃ and R₄ in Formula 3 may be combined to each other to form a chemical structure represented by the following Formula 4:

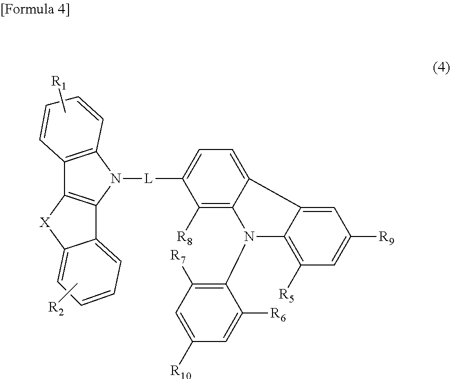

In Formula 4:

X may be an oxygen atom or a sulfur atom,

R₁ and R₂ may independently be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom,

R₅ to R₁₀ may independently be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom, and

L may be an arylene group.

Two or more of R₅ to R₈ in Formula 3 may be combined to each other to form a saturated or unsaturated ring.

### BRIEF DESCRIPTION OF THE DRAWINGS

Features will become apparent to those of ordinary skill in the art by describing in detail exemplary embodiments with reference to the attached drawing, in which:

FIG. 1 illustrates a schematic diagram of a structure of an organic EL device according to an example embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

A material for an organic EL device according to an example embodiment includes a triarylamine derivative having a heterocyclic part represented by the following Formula 3.

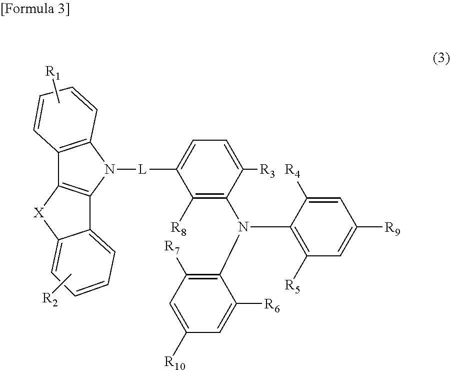

According to the present example embodiment, in the triarylamine derivative represented by Formula 3, X may be an oxygen atom or a sulfur atom. Thus, the triaryl amine derivative represented by Formula 3 according to an example embodiment may include an indolobenzofuranyl group or an indolobenzothiophenyl group, which may help lower an ionization energy of the organic EL device, e.g., when the material is used as a hole transport material.

According to the present example embodiment, in Formula 3, R₁ and R₂ may be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom. In addition, R₁ and R₂ may be the same or different.

R₁ and R₂ may be, for example, one of a methyl group, an ethyl group, a normal propyl group, an isopropyl group, a normal butyl group, a secondary butyl group, a tertiary butyl group, a phenyl group, a toluyl group, a biphenyl group, etc.

According to the present example embodiment, in Formula 3, R₃ to R₁₀ may be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, oxygen, sulfur, or a bond to another one of R₃ to R₁₀. In addition, R₃ to R₁₀ may be different from each other, and at least two of the substituents may be the same.

R₃ to R₁₀ may be, for example, one of a methyl group, an ethyl group, a normal propyl group, an isopropyl group, a normal butyl group, a secondary butyl group, a tertiary butyl group, a phenyl group, a toluyl group, a dimethylphenyl group, a trimethylphenyl group, a diphenylphenyl group, a triphenylphenyl group, a fluorophenyl group, a difluorophenyl group, a trifluorophenyl group, a tetrafluorophenyl group, a pentafluorophenyl group, a biphenyl group, a naphthyl group, a naphthyl phenyl group, a phenyl naphthyl group, a pyridyl group, a pyridyl phenyl group, a quinolyl group, a trimethylsilyl phenyl group, a triphenylsilyl phenyl group, etc.

According to the present example embodiment, in Formula 3, L may be an arylene group. The arylene group may be an arylene group having 6 to 18 ring carbon atoms. The material for an organic EL device according to an example embodiment may control the HOMO level appropriately by combining an indolobenzofuranyl group or an indolobenzothiophenyl group with a triarylamine part by using a connecting group.

L may be, for example, one of a phenylene group, a biphenylene group, a fluorenylene group, a naphthalene group, an anthracene group, etc.

In the triarylamine derivative represented by Formula 3 according to an example embodiment, the indolobenzofuranyl group or the indolobenzothiophenyl group is combined with the triarylamine part via the connecting group, which may provide an organic EL device capable of being driven at a low voltage, and having high efficiency and long life. In the triarylamine derivative represented by Formula 3 according to an example embodiment, the indolobenzofuranyl group or the indolobenzothiophenyl group is introduced instead of, for example, an indolobenzopyrrole group, and the HOMO level may thus be lowered. In addition, the material may be appropriately used as a host material of an emission layer, or a material of a stacking layer disposed between the emission layer and a positive electrode, for example, as a hole transport material. The properties of the device may be improved particularly in a blue emission region.

In an example embodiment, in Formula 3, two or more of R₃ to R₈ may be combined to each other and may form a saturated or unsaturated ring, which may provide an organic EL device capable of being driven at a low voltage, and having high efficiency and long life.

In an example embodiment, in the triarylamine derivative represented by Formula 3, R₃ and R₄ may be bonds combined to each other. For example, the triarylamine derivative represented by Formula 3 may have a chemical structure of the following Formula 4.

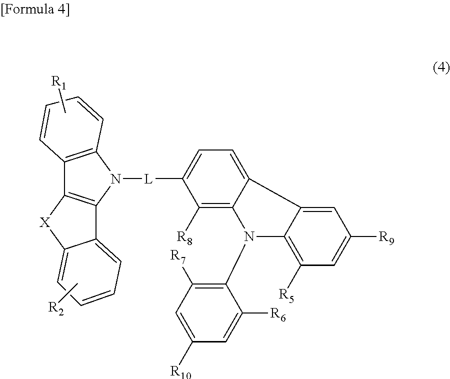

In Formula 4, X may be an oxygen atom or a sulfur atom. R₁ and R₂ may be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom. In addition, R₁ and R₂ may be the same or different. Particular examples of R₁ and R₂ are the same as described above, and detailed description thereof will be omitted.

In Formula 4, R₅ to R₁₀ may be a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom. R₅ to R₁₀ may be different from each other, and at least two substituents may be the same. In addition, particular examples of R₅ to R₁₀ are the same as described above, and detailed description thereof will be omitted.

In Formula 4, L may be an arylene group. The arylene group may be an arylene group having 6 to 18 ring carbon atoms. The material for an organic EL device according to an example embodiment may control the HOMO level appropriately by combining an indolobenzofuranyl group or an indolobenzothiophenyl group with a triarylamine part by using a connecting group. In addition, particular examples of L are the same as described above, and detailed description thereof will be omitted.

In the triarylamine derivative represented by Formula 4, R₃ and R₄ (of Formula 3) are combined to form a carbazole part. Thus, the triarylamine derivative represented by Formula 3 according to an example embodiment may have a structure in which an aryl group is combined to nitrogen at position 9 of a carbazolyl group, and carbon at position 2 is combined to the indolobenzofuranyl group or the indolobenzothiophenyl group via a divalent connecting group.

The material for an organic EL device according to an example embodiment may have a carbazolyl group, and hole transporting properties may be improved. In addition, the indolobenzofuranyl group or the indolobenzothiophenyl group included in the material may help lower ionization energy, and the material may be appropriated used as a hole transport material. By combining the indolobenzofuranyl group or the indolobenzothiophenyl group with the carbazolyl group via a connecting group, the HOMO level of the material may be appropriately controlled. Therefore, the HOMO level of the triarylamine derivative represented by Formula 3 according to an example embodiment may be lowered, and the material may be appropriately used as a host material of an emission layer, or as a material of a stacking layer disposed between the emission layer and a positive electrode, for example, as a hole transport material. An organic EL device capable of being driven at a low voltage and having high efficiency and long life may be manufactured. The properties of the device may be improved particularly in a blue emission region.

In addition, Formula 3, two or more of R₅ to R₈ may be combined to each other to form a saturated or unsaturated ring. The combination of two or more of R₅ to R₈ to each to form a saturated or unsaturated ring may help provide an organic EL device capable of being driven at a low voltage, and having high efficiency and long life.

The material for an organic EL device according to an example embodiment may be a material having a structure illustrated in the following Formula 5.

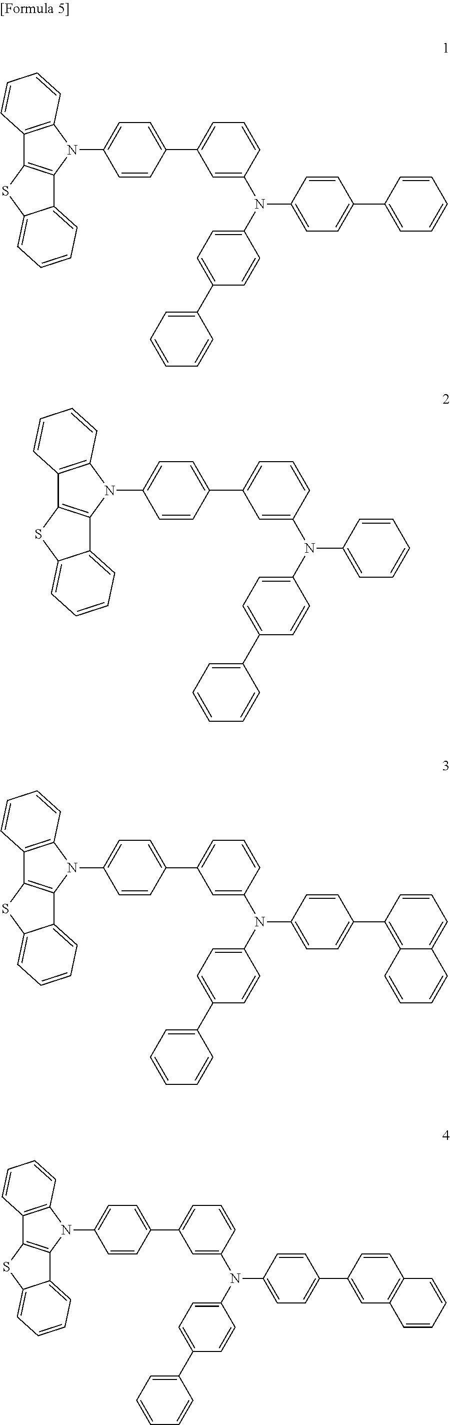 [SMILES: c1ccc(-c2ccc(N(c3ccc(-c4ccccc4)cc3)c3cccc(-c4ccc(-n5c6ccccc6c6sc7ccccc7c65)cc4)c3)cc2)cc1 ; c1ccc(-c2ccc(N(c3ccccc3)c3cccc(-c4ccc(-n5c6ccccc6c6sc7ccccc7c65)cc4)c3)cc2)cc1 ; c1ccc(-c2ccc(N(c3ccc(-c4cccc5ccccc45)cc3)c3cccc(-c4ccc(-n5c6ccccc6c6sc7ccccc7c65)cc4)c3)cc2)cc1 ; c1ccc(-c2ccc(N(c3ccc(-c4ccc5ccccc5c4)cc3)c3cccc(-c4ccc(-n5c6ccccc6c6sc7ccccc7c65)cc4)c3)cc2)cc1]

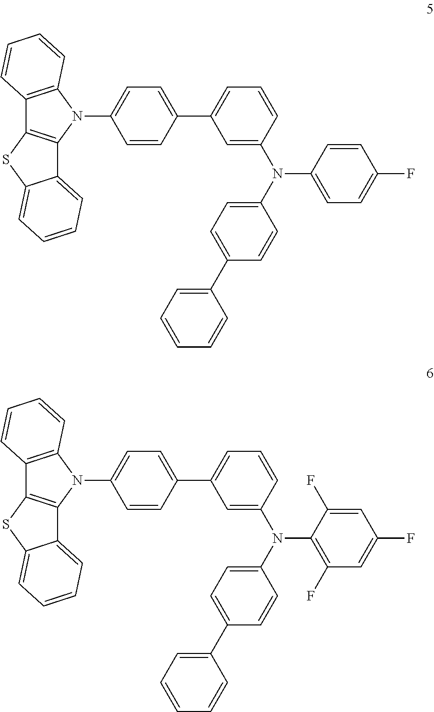 [SMILES: Fc1ccc(N(c2ccc(-c3ccccc3)cc2)c2cccc(-c3ccc(-n4c5ccccc5c5sc6ccccc6c54)cc3)c2)cc1 ; Fc1cc(F)c(N(c2ccc(-c3ccccc3)cc2)c2cccc(-c3ccc(-n4c5ccccc5c5sc6ccccc6c54)cc3)c2)c(F)c1]

The material for an organic EL device according to an example embodiment may be a material having a structure illustrated in the following Formula 6.

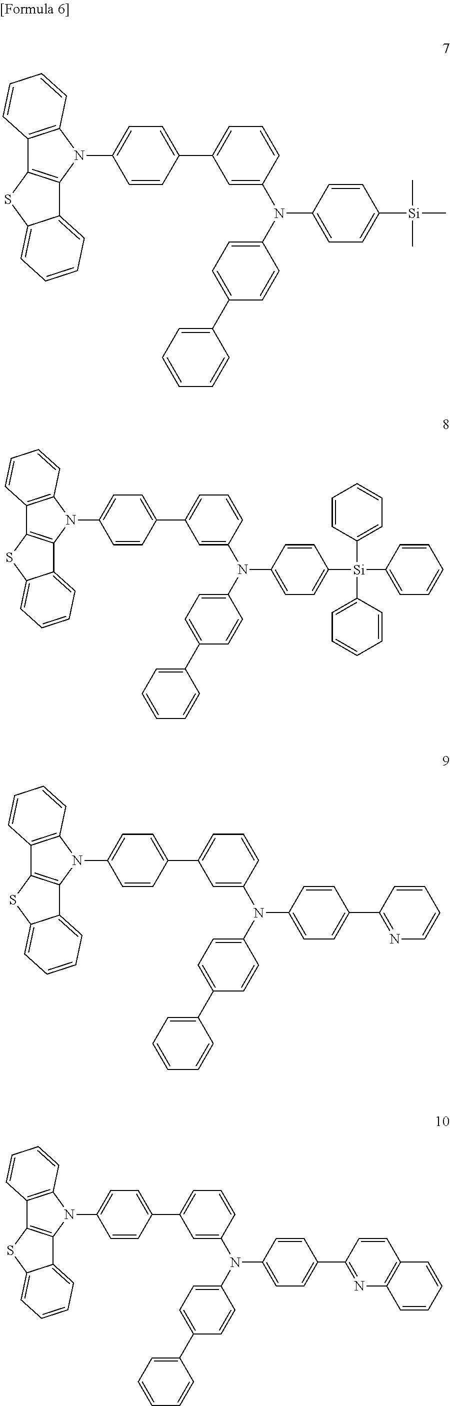 [SMILES: C[Si](C)(C)c1ccc(N(c2ccc(-c3ccccc3)cc2)c2cccc(-c3ccc(-n4c5ccccc5c5sc6ccccc6c54)cc3)c2)cc1 ; c1ccc(-c2ccc(N(c3ccc([Si](c4ccccc4)(c4ccccc4)c4ccccc4)cc3)c3cccc(-c4ccc(-n5c6ccccc6c6sc7ccccc7c65)cc4)c3)cc2)cc1 ; c1ccc(-c2ccc(N(c3ccc(-c4ccccn4)cc3)c3cccc(-c4ccc(-n5c6ccccc6c6sc7ccccc7c65)cc4)c3)cc2)cc1 ; c1ccc(-c2ccc(N(c3ccc(-c4ccc5ccccc5n4)cc3)c3cccc(-c4ccc(-n5c6ccccc6c6sc7ccccc7c65)cc4)c3)cc2)cc1]

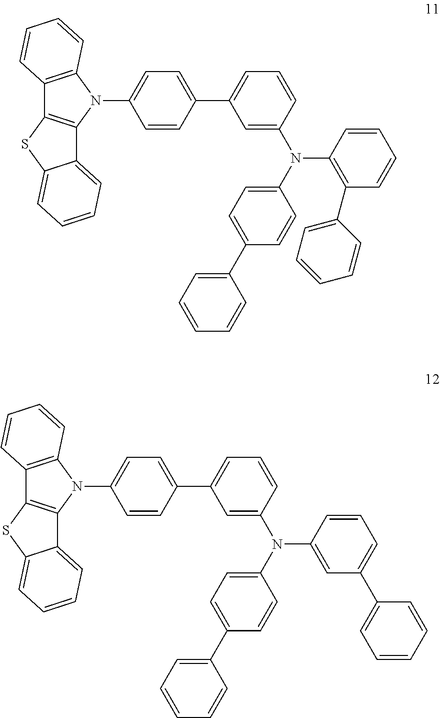 [SMILES: c1ccc(-c2ccc(N(c3cccc(-c4ccc(-n5c6ccccc6c6sc7ccccc7c65)cc4)c3)c3ccccc3-c3ccccc3)cc2)cc1 ; c1ccc(-c2ccc(N(c3cccc(-c4ccccc4)c3)c3cccc(-c4ccc(-n5c6ccccc6c6sc7ccccc7c65)cc4)c3)cc2)cc1]

The material for an organic EL device according to an example embodiment may be a material having a structure illustrated in the following Formula 7.

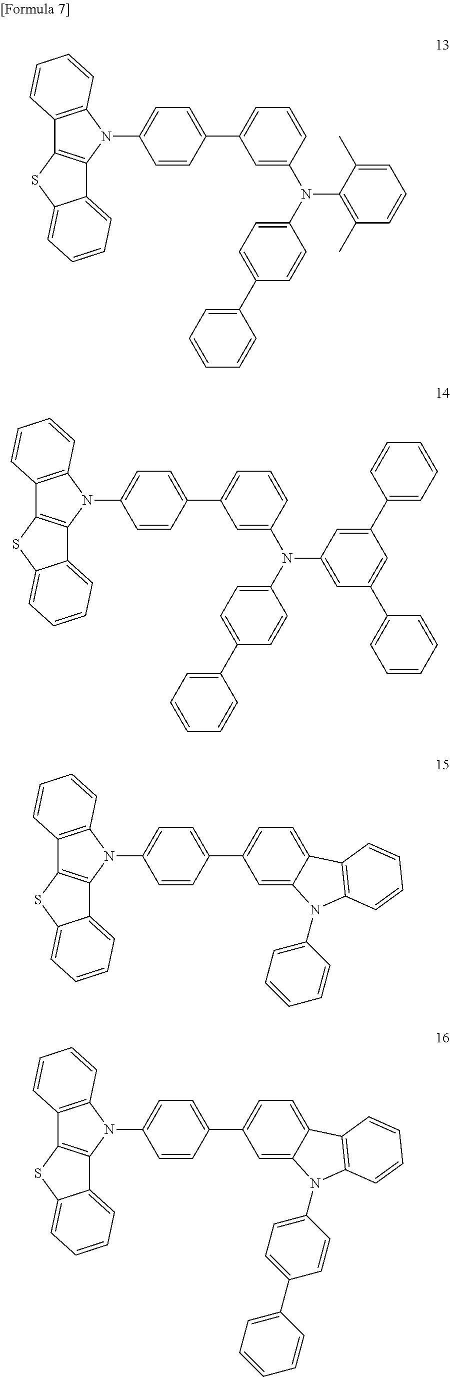 [SMILES: Cc1cccc(C)c1N(c1ccc(-c2ccccc2)cc1)c1cccc(-c2ccc(-n3c4ccccc4c4sc5ccccc5c43)cc2)c1 ; c1ccc(-c2ccc(N(c3cccc(-c4ccc(-n5c6ccccc6c6sc7ccccc7c65)cc4)c3)c3cc(-c4ccccc4)cc(-c4ccccc4)c3)cc2)cc1 ; c1ccc(-n2c3ccccc3c3ccc(-c4ccc(-n5c6ccccc6c6sc7ccccc7c65)cc4)cc32)cc1 ; c1ccc(-c2ccc(-n3c4ccccc4c4ccc(-c5ccc(-n6c7ccccc7c7sc8ccccc8c76)cc5)cc43)cc2)cc1]

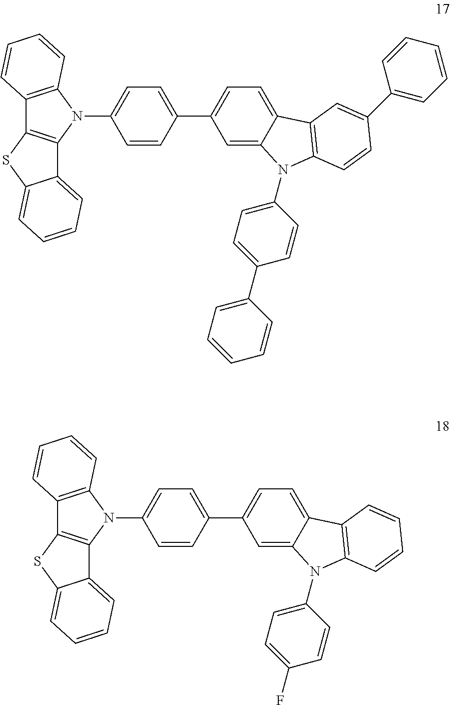 [SMILES: c1ccc(-c2ccc(-n3c4ccc(-c5ccccc5)cc4c4ccc(-c5ccc(-n6c7ccccc7c7sc8ccccc8c76)cc5)cc43)cc2)cc1 ; Fc1ccc(-n2c3ccccc3c3ccc(-c4ccc(-n5c6ccccc6c6sc7ccccc7c65)cc4)cc32)cc1]

The material for an organic EL device according to an example embodiment may be a material having a structure illustrated in the following Formula 8.

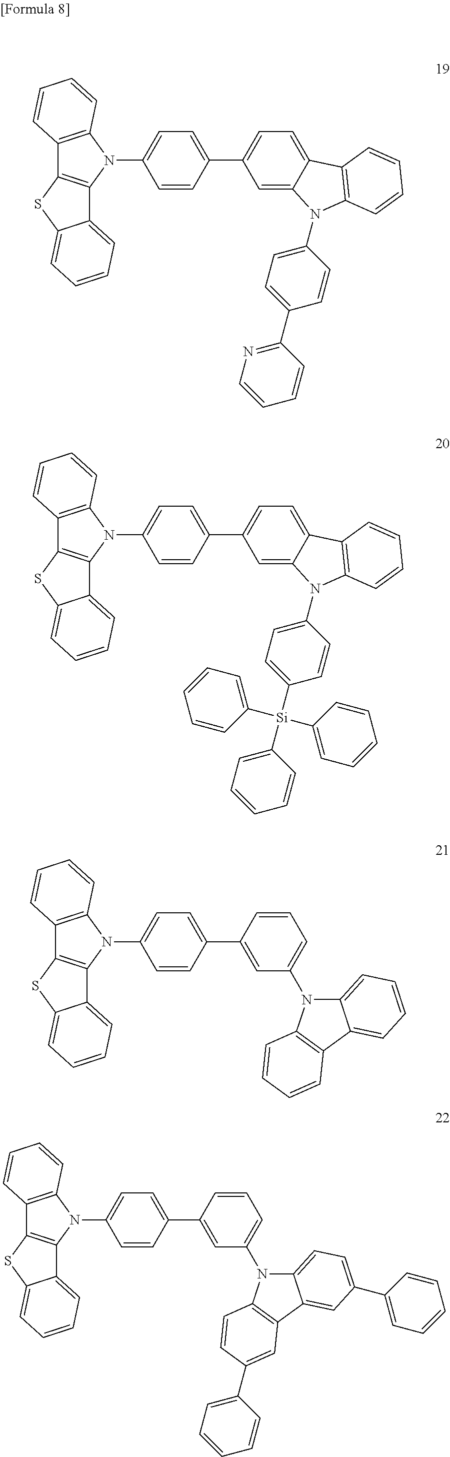 [SMILES: c1ccc(-c2ccc(-n3c4ccccc4c4ccc(-c5ccc(-n6c7ccccc7c7sc8ccccc8c76)cc5)cc43)cc2)nc1 ; c1ccc([Si](c2ccccc2)(c2ccccc2)c2ccc(-n3c4ccccc4c4ccc(-c5ccc(-n6c7ccccc7c7sc8ccccc8c76)cc5)cc43)cc2)cc1 ; c1cc(-c2ccc(-n3c4ccccc4c4sc5ccccc5c43)cc2)cc(-n2c3ccccc3c3ccccc32)c1 ; c1ccc(-c2ccc3c(c2)c2cc(-c4ccccc4)ccc2n3-c2cccc(-c3ccc(-n4c5ccccc5c5sc6ccccc6c54)cc3)c2)cc1]

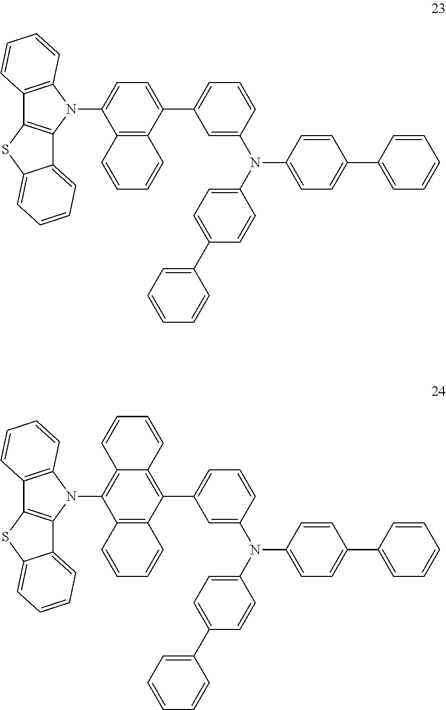 [SMILES: c1ccc(-c2ccc(N(c3ccc(-c4ccccc4)cc3)c3cccc(-c4ccc(-n5c6ccccc6c6sc7ccccc7c65)c5ccccc45)c3)cc2)cc1 ; c1ccc(-c2ccc(N(c3ccc(-c4ccccc4)cc3)c3cccc(-c4c5ccccc5c(-n5c6ccccc6c6sc7ccccc7c65)c5ccccc45)c3)cc2)cc1]

The material for an organic EL device according to an example embodiment may be a material having a structure illustrated in the following Formula 9.

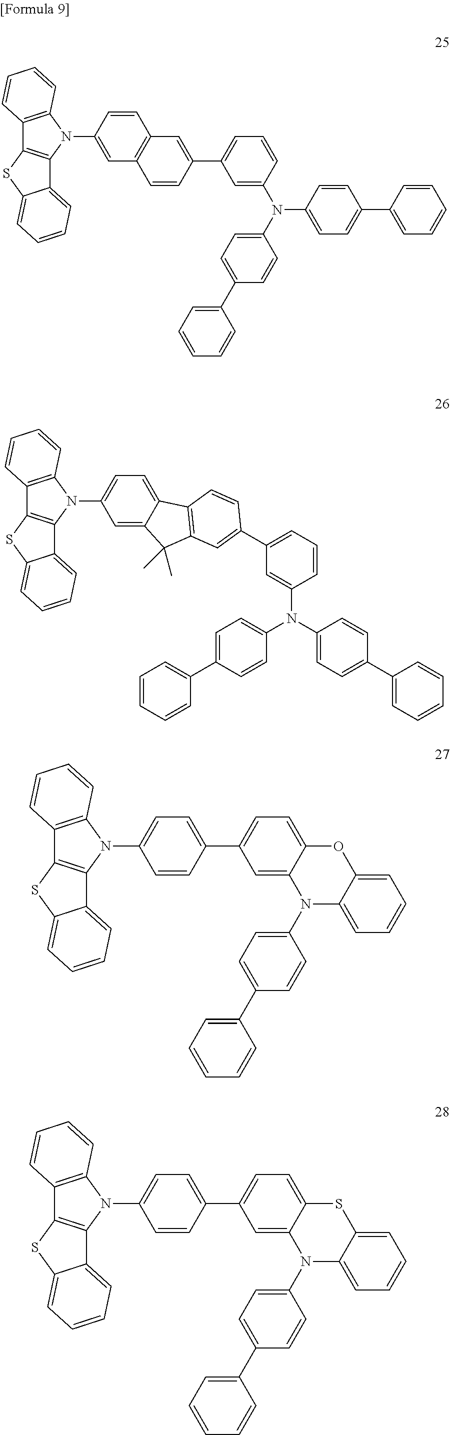 [SMILES: c1ccc(-c2ccc(N(c3ccc(-c4ccccc4)cc3)c3cccc(-c4ccc5cc(-n6c7ccccc7c7sc8ccccc8c76)ccc5c4)c3)cc2)cc1 ; CC1(C)c2cc(-c3cccc(N(c4ccc(-c5ccccc5)cc4)c4ccc(-c5ccccc5)cc4)c3)ccc2-c2ccc(-n3c4ccccc4c4sc5ccccc5c43)cc21 ; c1ccc(-c2ccc(N3c4ccccc4Oc4ccc(-c5ccc(-n6c7ccccc7c7sc8ccccc8c76)cc5)cc43)cc2)cc1 ; c1ccc(-c2ccc(N3c4ccccc4Sc4ccc(-c5ccc(-n6c7ccccc7c7sc8ccccc8c76)cc5)cc43)cc2)cc1]

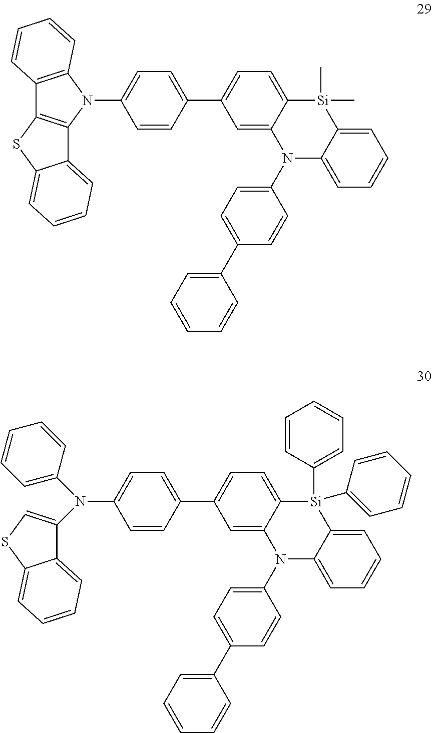 [SMILES: C[Si]1(C)c2ccccc2N(c2ccc(-c3ccccc3)cc2)c2cc(-c3ccc(-n4c5ccccc5c5sc6ccccc6c54)cc3)ccc21 ; c1ccc(-c2ccc(N3c4ccccc4[Si](c4ccccc4)(c4ccccc4)c4ccc(-c5ccc(N(c6ccccc6)c6csc7ccccc67)cc5)cc43)cc2)cc1]

The material for an organic EL device according to an example embodiment may be a material having a structure illustrated in the following Formula 10.

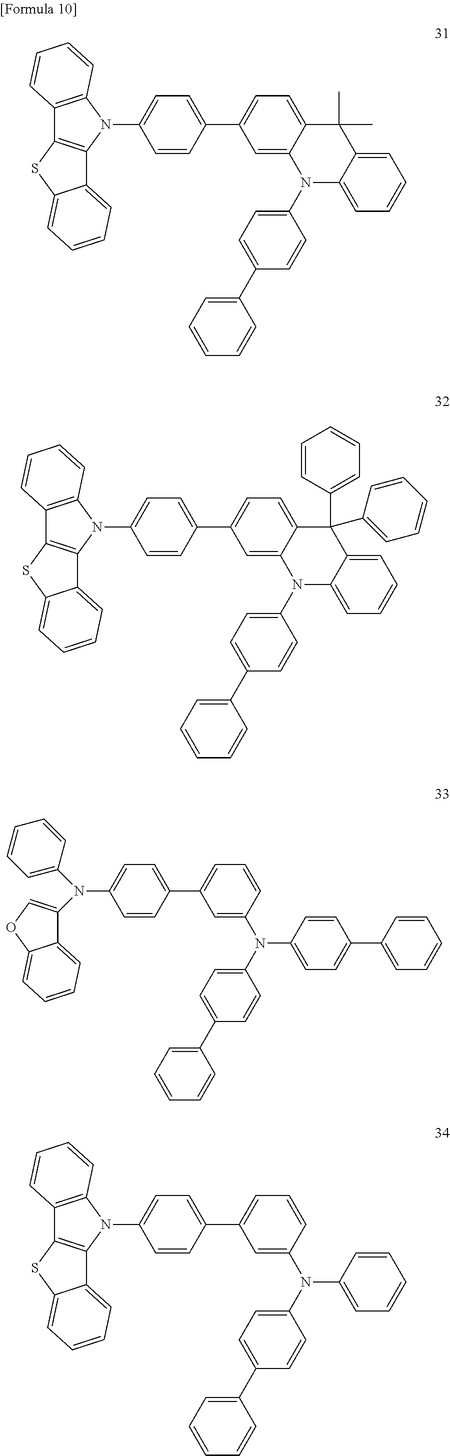 [SMILES: CC1(C)c2ccccc2N(c2ccc(-c3ccccc3)cc2)c2cc(-c3ccc(-n4c5ccccc5c5sc6ccccc6c54)cc3)ccc21 ; c1ccc(-c2ccc(N3c4ccccc4C(c4ccccc4)(c4ccccc4)c4ccc(-c5ccc(-n6c7ccccc7c7sc8ccccc8c76)cc5)cc43)cc2)cc1 ; c1ccc(-c2ccc(N(c3ccc(-c4ccccc4)cc3)c3cccc(-c4ccc(N(c5ccccc5)c5coc6ccccc56)cc4)c3)cc2)cc1 ; c1ccc(-c2ccc(N(c3ccccc3)c3cccc(-c4ccc(-n5c6ccccc6c6sc7ccccc7c65)cc4)c3)cc2)cc1]

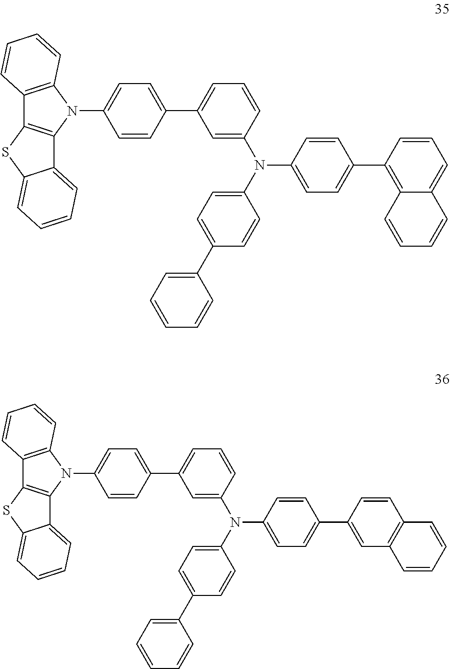 [SMILES: c1ccc(-c2ccc(N(c3ccc(-c4cccc5ccccc45)cc3)c3cccc(-c4ccc(-n5c6ccccc6c6sc7ccccc7c65)cc4)c3)cc2)cc1 ; c1ccc(-c2ccc(N(c3ccc(-c4ccc5ccccc5c4)cc3)c3cccc(-c4ccc(-n5c6ccccc6c6sc7ccccc7c65)cc4)c3)cc2)cc1]

The material for an organic EL device according to an example embodiment may be a material having a structure illustrated in the following Formula 11.

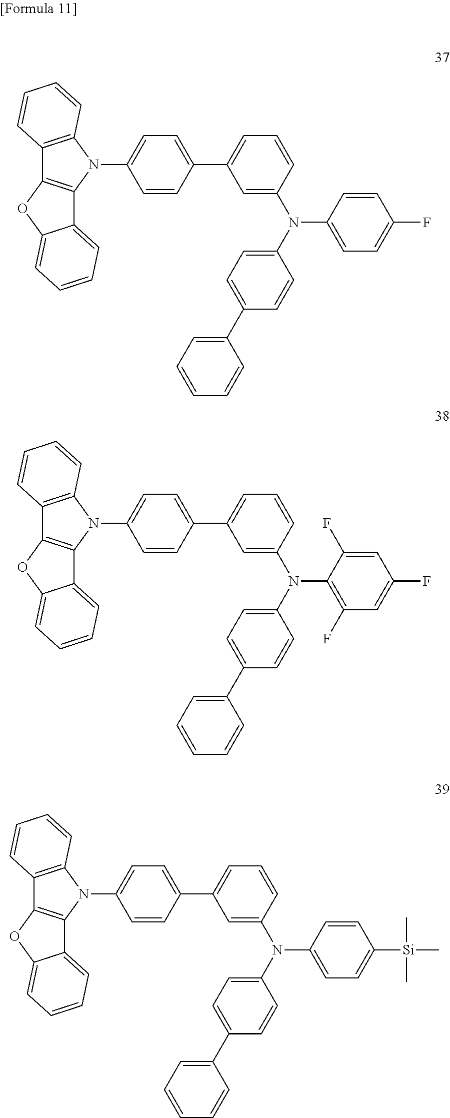 [SMILES: Fc1ccc(N(c2ccc(-c3ccccc3)cc2)c2cccc(-c3ccc(-n4c5ccccc5c5oc6ccccc6c54)cc3)c2)cc1 ; Fc1cc(F)c(N(c2ccc(-c3ccccc3)cc2)c2cccc(-c3ccc(-n4c5ccccc5c5oc6ccccc6c54)cc3)c2)c(F)c1 ; C[Si](C)(C)c1ccc(N(c2ccc(-c3ccccc3)cc2)c2cccc(-c3ccc(-n4c5ccccc5c5oc6ccccc6c54)cc3)c2)cc1]

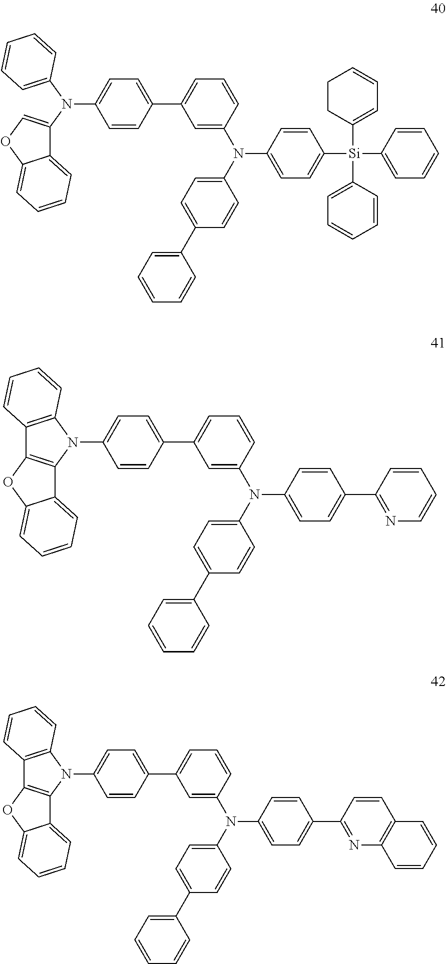 [SMILES: C1=CCCC([Si](c2ccccc2)(c2ccccc2)c2ccc(N(c3ccc(-c4ccccc4)cc3)c3cccc(-c4ccc(N(c5ccccc5)c5coc6ccccc56)cc4)c3)cc2)=C1 ; c1ccc(-c2ccc(N(c3ccc(-c4ccccn4)cc3)c3cccc(-c4ccc(-n5c6ccccc6c6oc7ccccc7c65)cc4)c3)cc2)cc1 ; c1ccc(-c2ccc(N(c3ccc(-c4ccc5ccccc5n4)cc3)c3cccc(-c4ccc(-n5c6ccccc6c6oc7ccccc7c65)cc4)c3)cc2)cc1]

The material for an organic EL device according to an example embodiment may be a material having a structure illustrated in the following Formula 12.

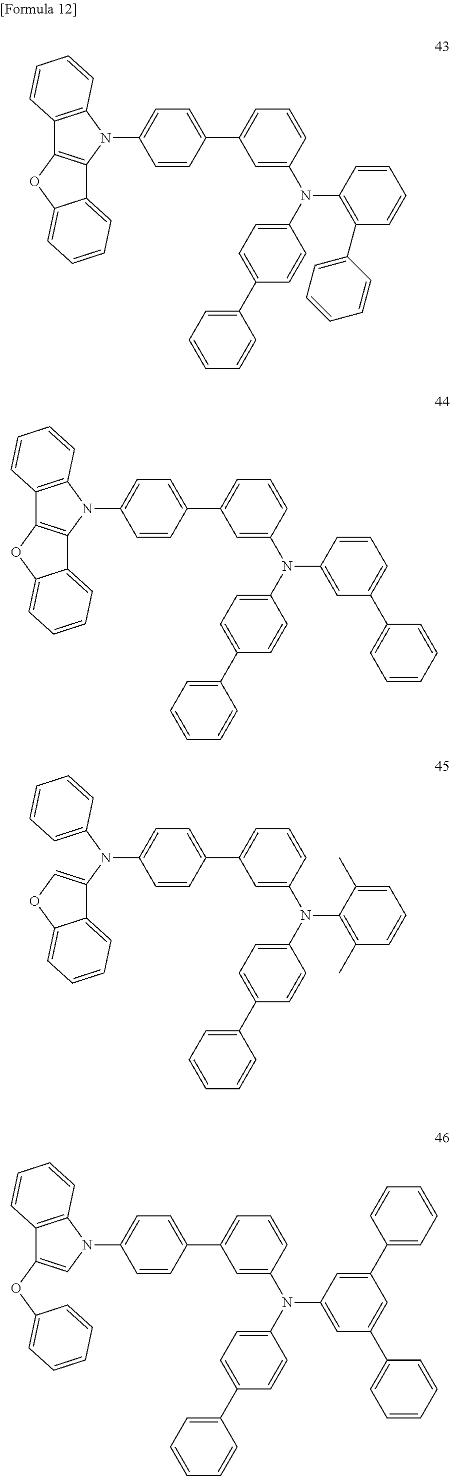 [SMILES: c1ccc(-c2ccc(N(c3cccc(-c4ccc(-n5c6ccccc6c6oc7ccccc7c65)cc4)c3)c3ccccc3-c3ccccc3)cc2)cc1 ; c1ccc(-c2ccc(N(c3cccc(-c4ccccc4)c3)c3cccc(-c4ccc(-n5c6ccccc6c6oc7ccccc7c65)cc4)c3)cc2)cc1 ; Cc1cccc(C)c1N(c1ccc(-c2ccccc2)cc1)c1cccc(-c2ccc(N(c3ccccc3)c3coc4ccccc34)cc2)c1 ; c1ccc(Oc2cn(-c3ccc(-c4cccc(N(c5ccc(-c6ccccc6)cc5)c5cc(-c6ccccc6)cc(-c6ccccc6)c5)c4)cc3)c3ccccc23)cc1]

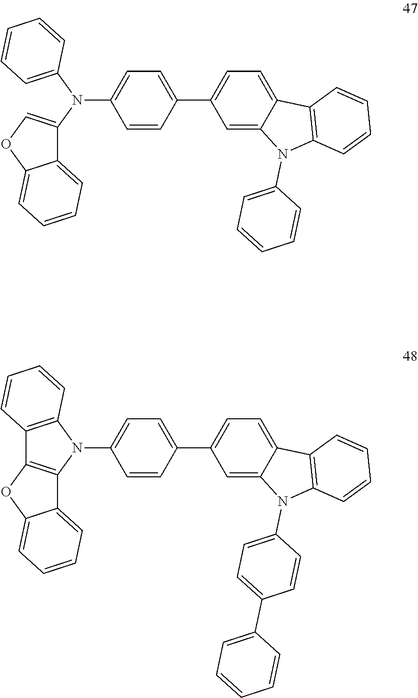 [SMILES: c1ccc(N(c2ccc(-c3ccc4c5ccccc5n(-c5ccccc5)c4c3)cc2)c2coc3ccccc23)cc1 ; c1ccc(-c2ccc(-n3c4ccccc4c4ccc(-c5ccc(-n6c7ccccc7c7oc8ccccc8c76)cc5)cc43)cc2)cc1]

The material for an organic EL device according to an example embodiment may be a material having a structure illustrated in the following Formula 13.

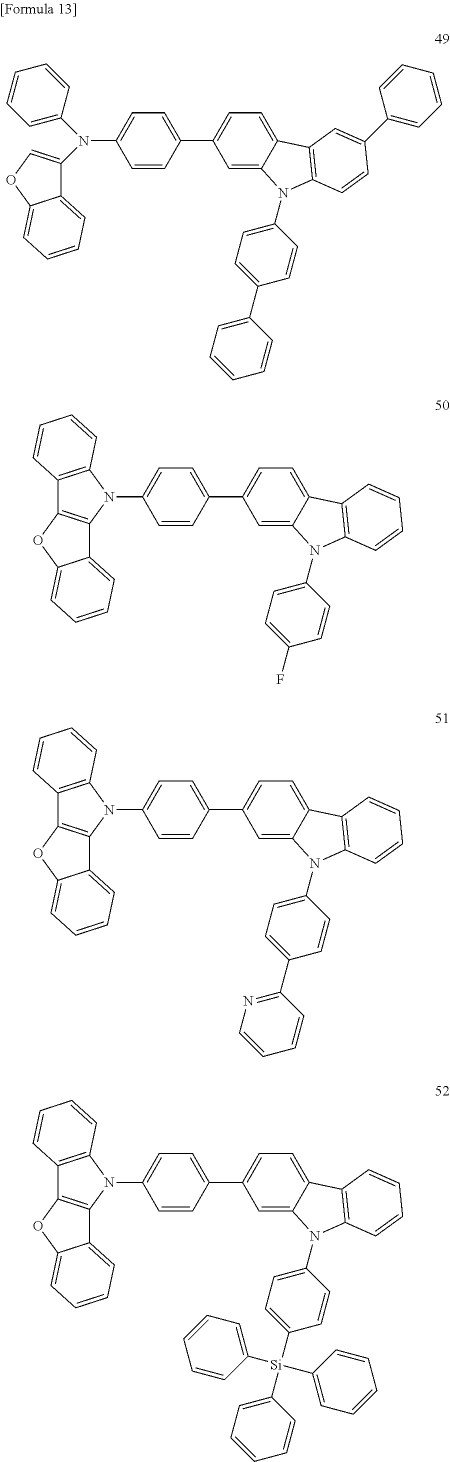 [SMILES: c1ccc(-c2ccc(-n3c4ccc(-c5ccccc5)cc4c4ccc(-c5ccc(N(c6ccccc6)c6coc7ccccc67)cc5)cc43)cc2)cc1 ; Fc1ccc(-n2c3ccccc3c3ccc(-c4ccc(-n5c6ccccc6c6oc7ccccc7c65)cc4)cc32)cc1 ; c1ccc(-c2ccc(-n3c4ccccc4c4ccc(-c5ccc(-n6c7ccccc7c7oc8ccccc8c76)cc5)cc43)cc2)nc1 ; c1ccc([Si](c2ccccc2)(c2ccccc2)c2ccc(-n3c4ccccc4c4ccc(-c5ccc(-n6c7ccccc7c7oc8ccccc8c76)cc5)cc43)cc2)cc1]

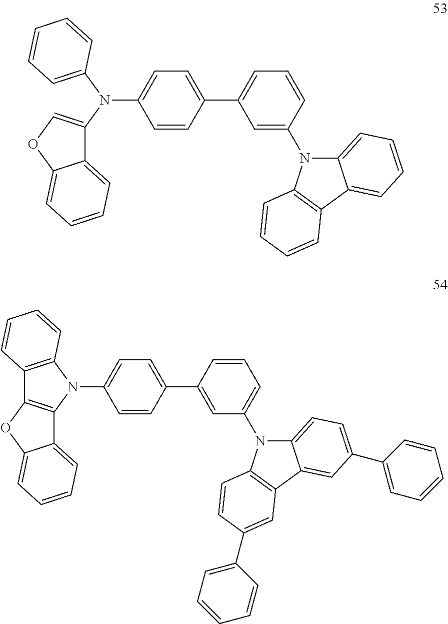 [SMILES: c1ccc(N(c2ccc(-c3cccc(-n4c5ccccc5c5ccccc54)c3)cc2)c2coc3ccccc23)cc1 ; c1ccc(-c2ccc3c(c2)c2cc(-c4ccccc4)ccc2n3-c2cccc(-c3ccc(-n4c5ccccc5c5oc6ccccc6c54)cc3)c2)cc1]

The material for an organic EL device according to an example embodiment may be a material having a structure illustrated in the following Formula 14.

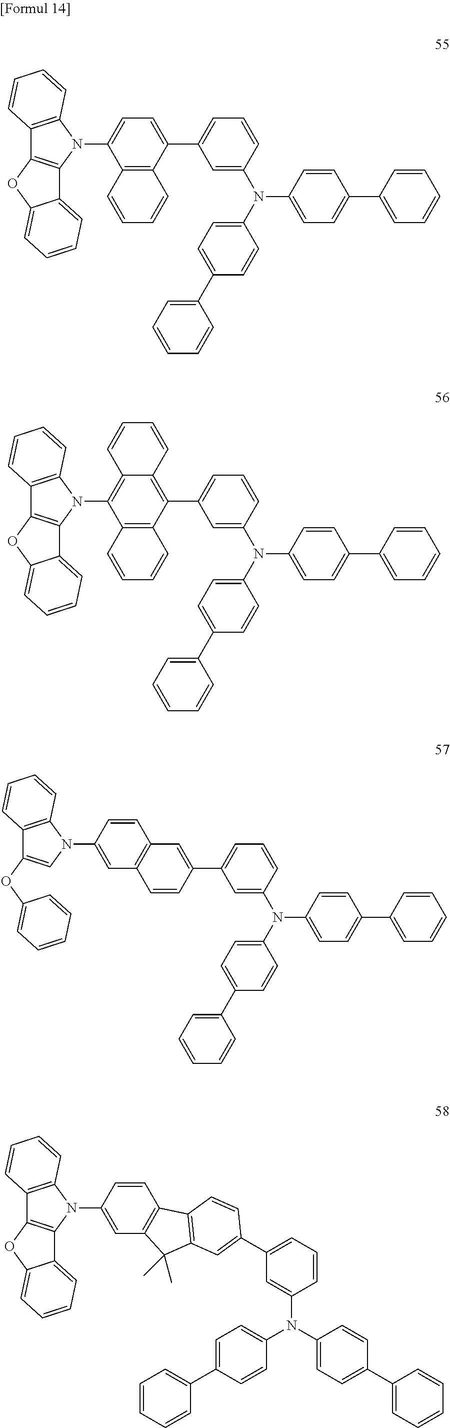 [SMILES: c1ccc(-c2ccc(N(c3ccc(-c4ccccc4)cc3)c3cccc(-c4ccc(-n5c6ccccc6c6oc7ccccc7c65)c5ccccc45)c3)cc2)cc1 ; c1ccc(-c2ccc(N(c3ccc(-c4ccccc4)cc3)c3cccc(-c4c5ccccc5c(-n5c6ccccc6c6oc7ccccc7c65)c5ccccc45)c3)cc2)cc1 ; c1ccc(Oc2cn(-c3ccc4cc(-c5cccc(N(c6ccc(-c7ccccc7)cc6)c6ccc(-c7ccccc7)cc6)c5)ccc4c3)c3ccccc23)cc1 ; CC1(C)c2cc(-c3cccc(N(c4ccc(-c5ccccc5)cc4)c4ccc(-c5ccccc5)cc4)c3)ccc2-c2ccc(-n3c4ccccc4c4oc5ccccc5c43)cc21]

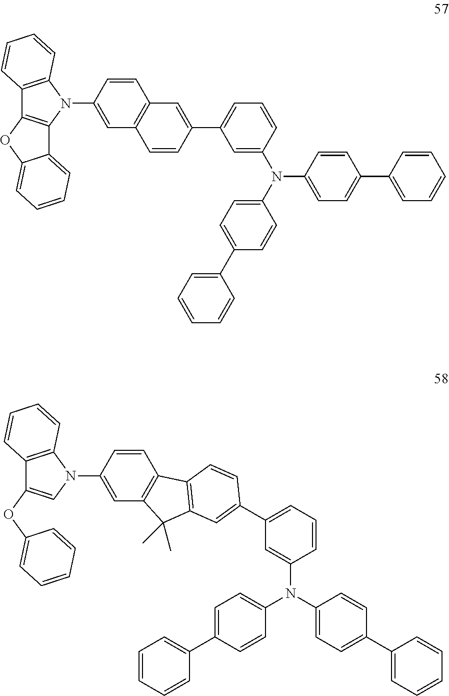 [SMILES: c1ccc(-c2ccc(N(c3ccc(-c4ccccc4)cc3)c3cccc(-c4ccc5cc(-n6c7ccccc7c7oc8ccccc8c76)ccc5c4)c3)cc2)cc1 ; CC1(C)c2cc(-c3cccc(N(c4ccc(-c5ccccc5)cc4)c4ccc(-c5ccccc5)cc4)c3)ccc2-c2ccc(-n3cc(Oc4ccccc4)c4ccccc43)cc21]

The material for an organic EL device according to an example embodiment may be a material having a structure illustrated in the following Formula 15.

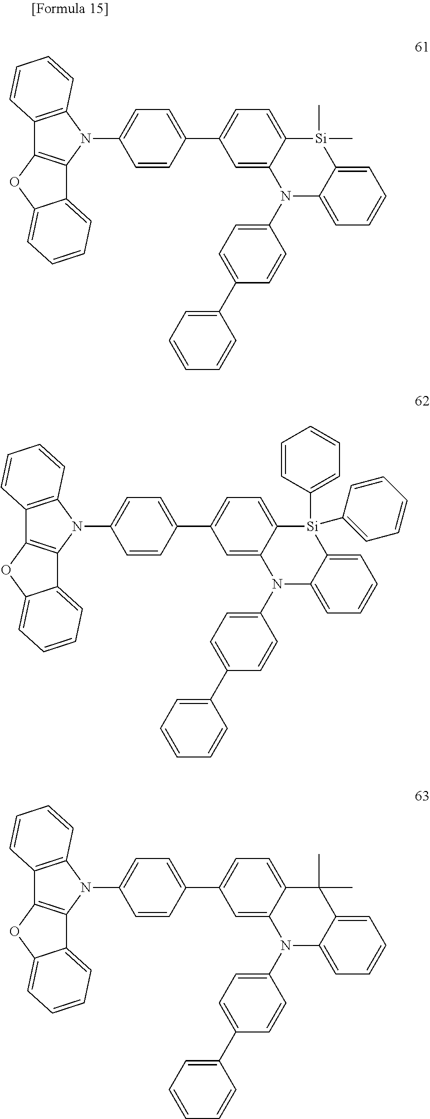 [SMILES: C[Si]1(C)c2ccccc2N(c2ccc(-c3ccccc3)cc2)c2cc(-c3ccc(-n4c5ccccc5c5oc6ccccc6c54)cc3)ccc21 ; c1ccc(-c2ccc(N3c4ccccc4[Si](c4ccccc4)(c4ccccc4)c4ccc(-c5ccc(-n6c7ccccc7c7oc8ccccc8c76)cc5)cc43)cc2)cc1 ; CC1(C)c2ccccc2N(c2ccc(-c3ccccc3)cc2)c2cc(-c3ccc(-n4c5ccccc5c5oc6ccccc6c54)cc3)ccc21]

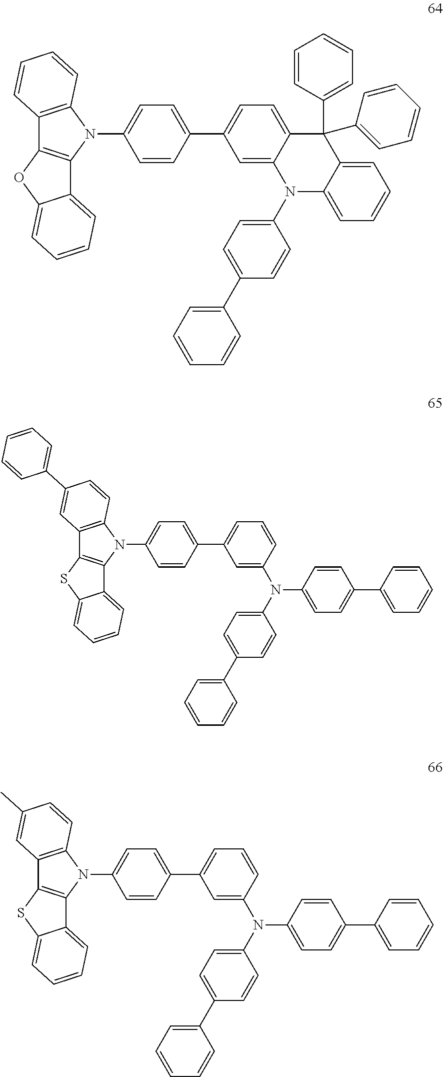 [SMILES: c1ccc(-c2ccc(N3c4ccccc4C(c4ccccc4)(c4ccccc4)c4ccc(-c5ccc(-n6c7ccccc7c7oc8ccccc8c76)cc5)cc43)cc2)cc1 ; c1ccc(-c2ccc(N(c3ccc(-c4ccccc4)cc3)c3cccc(-c4ccc(-n5c6ccc(-c7ccccc7)cc6c6sc7ccccc7c65)cc4)c3)cc2)cc1 ; Cc1ccc2c(c1)c1sc3ccccc3c1n2-c1ccc(-c2cccc(N(c3ccc(-c4ccccc4)cc3)c3ccc(-c4ccccc4)cc3)c2)cc1]

The material for an organic EL device according to an example embodiment may be a material having a structure illustrated in the following Formula 16.

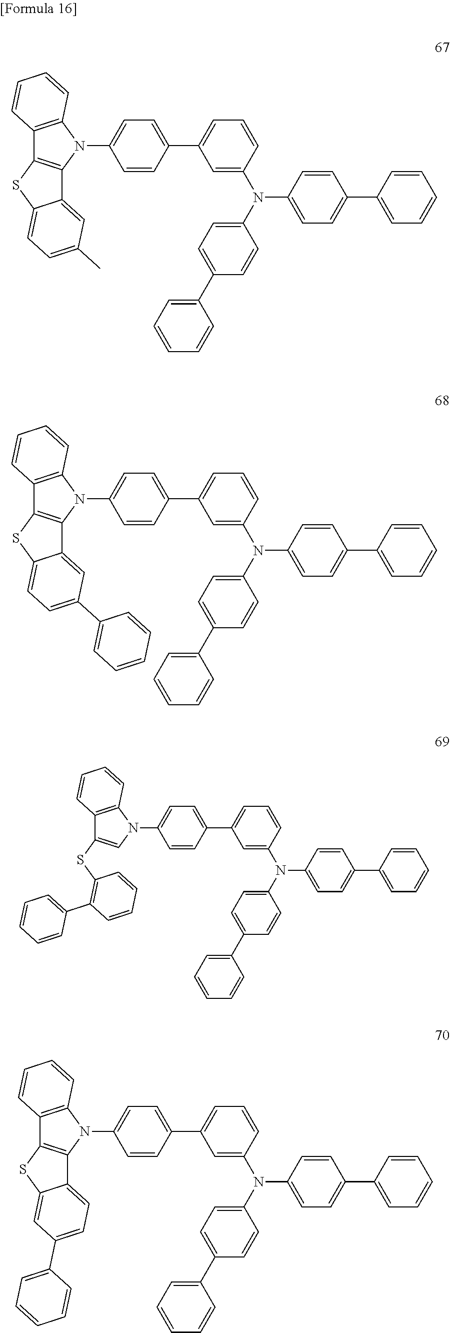 [SMILES: Cc1ccc2sc3c4ccccc4n(-c4ccc(-c5cccc(N(c6ccc(-c7ccccc7)cc6)c6ccc(-c7ccccc7)cc6)c5)cc4)c3c2c1 ; c1ccc(-c2ccc(N(c3ccc(-c4ccccc4)cc3)c3cccc(-c4ccc(-n5c6ccccc6c6sc7ccc(-c8ccccc8)cc7c65)cc4)c3)cc2)cc1 ; c1ccc(-c2ccc(N(c3ccc(-c4ccccc4)cc3)c3cccc(-c4ccc(-n5cc(Sc6ccccc6-c6ccccc6)c6ccccc65)cc4)c3)cc2)cc1 ; c1ccc(-c2ccc(N(c3ccc(-c4ccccc4)cc3)c3cccc(-c4ccc(-n5c6ccccc6c6sc7cc(-c8ccccc8)ccc7c65)cc4)c3)cc2)cc1]

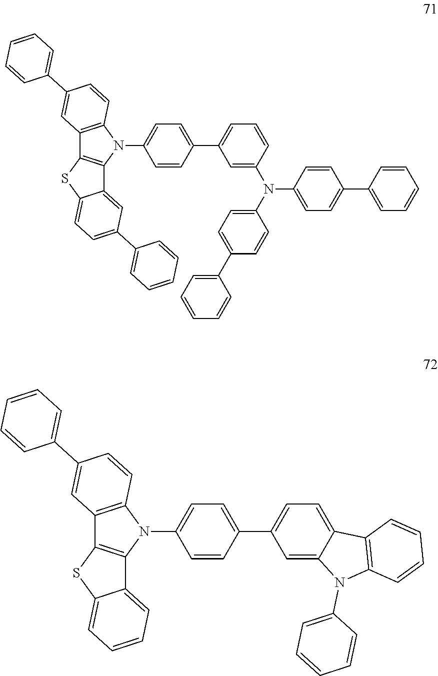 [SMILES: c1ccc(-c2ccc(N(c3ccc(-c4ccccc4)cc3)c3cccc(-c4ccc(-n5c6ccc(-c7ccccc7)cc6c6sc7ccc(-c8ccccc8)cc7c65)cc4)c3)cc2)cc1 ; c1ccc(-c2ccc3c(c2)c2sc4ccccc4c2n3-c2ccc(-c3ccc4c5ccccc5n(-c5ccccc5)c4c3)cc2)cc1]

The material for an organic EL device according to an example embodiment may be a material having a structure illustrated in the following Formula 17.

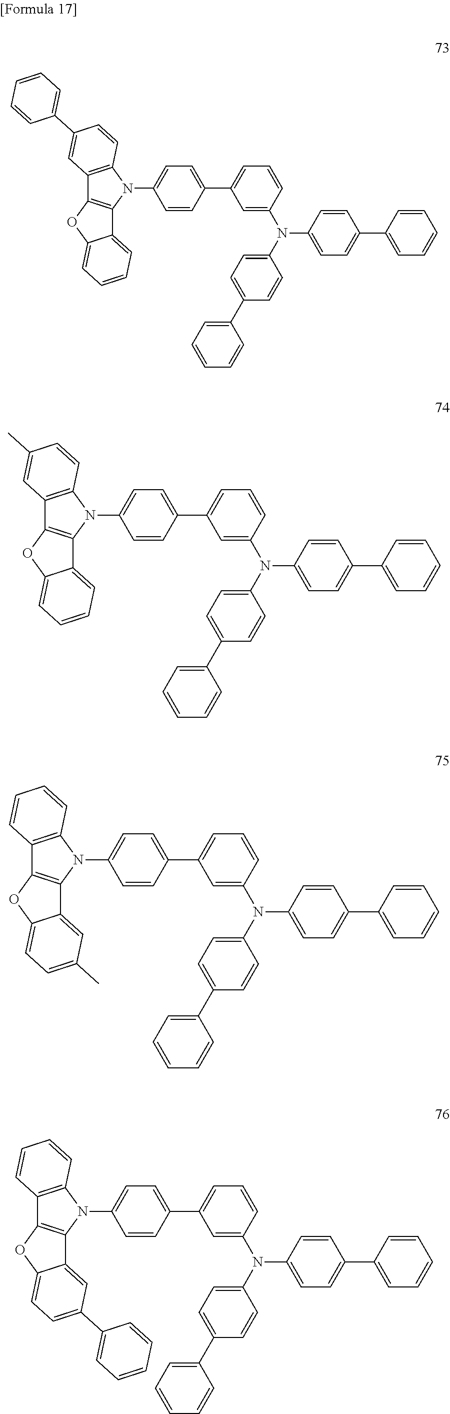 [SMILES: c1ccc(-c2ccc(N(c3ccc(-c4ccccc4)cc3)c3cccc(-c4ccc(-n5c6ccc(-c7ccccc7)cc6c6oc7ccccc7c65)cc4)c3)cc2)cc1 ; Cc1ccc2c(c1)c1oc3ccccc3c1n2-c1ccc(-c2cccc(N(c3ccc(-c4ccccc4)cc3)c3ccc(-c4ccccc4)cc3)c2)cc1 ; Cc1ccc2oc3c4ccccc4n(-c4ccc(-c5cccc(N(c6ccc(-c7ccccc7)cc6)c6ccc(-c7ccccc7)cc6)c5)cc4)c3c2c1 ; c1ccc(-c2ccc(N(c3ccc(-c4ccccc4)cc3)c3cccc(-c4ccc(-n5c6ccccc6c6oc7ccc(-c8ccccc8)cc7c65)cc4)c3)cc2)cc1]

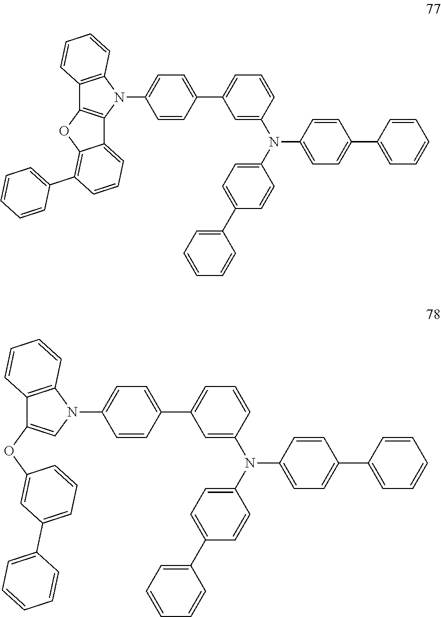 [SMILES: c1ccc(-c2ccc(N(c3ccc(-c4ccccc4)cc3)c3cccc(-c4ccc(-n5c6ccccc6c6oc7c(-c8ccccc8)cccc7c65)cc4)c3)cc2)cc1 ; c1ccc(-c2ccc(N(c3ccc(-c4ccccc4)cc3)c3cccc(-c4ccc(-n5cc(Oc6cccc(-c7ccccc7)c6)c6ccccc65)cc4)c3)cc2)cc1]

The material for an organic EL device according to an example embodiment may be a material having a structure illustrated in the following Formula 18.

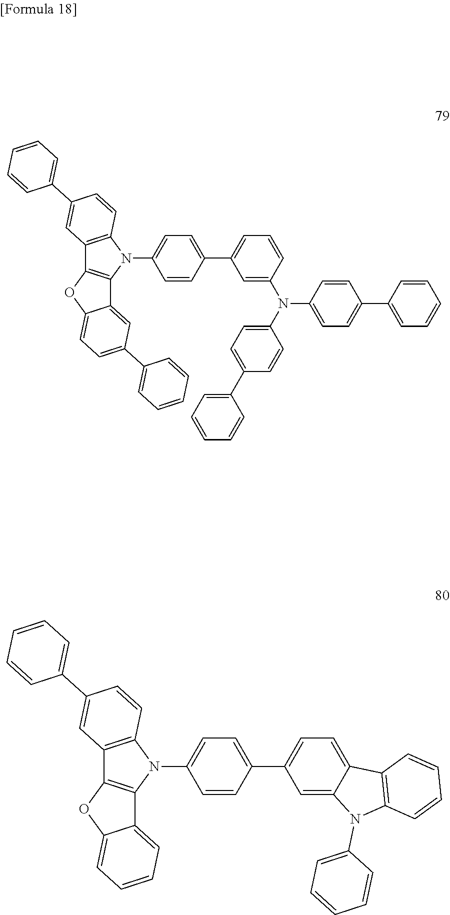 [SMILES: c1ccc(-c2ccc(N(c3ccc(-c4ccccc4)cc3)c3cccc(-c4ccc(-n5c6ccc(-c7ccccc7)cc6c6oc7ccc(-c8ccccc8)cc7c65)cc4)c3)cc2)cc1 ; c1ccc(-c2ccc3c(c2)c2oc4ccccc4c2n3-c2ccc(-c3ccc4c5ccccc5n(-c5ccccc5)c4c3)cc2)cc1]

The material for an organic EL device according to an example embodiment may be appropriately used in an emission layer of an organic device. In addition, the triarylamine derivative represented by Formula 3 according to an example embodiment may be used in a layer, for example, one of a stack of layers, disposed between an emission layer and a positive electrode, which may help improve hole transporting properties, lower the driving voltage, and increase efficiency of the device.

(Organic EL Device)

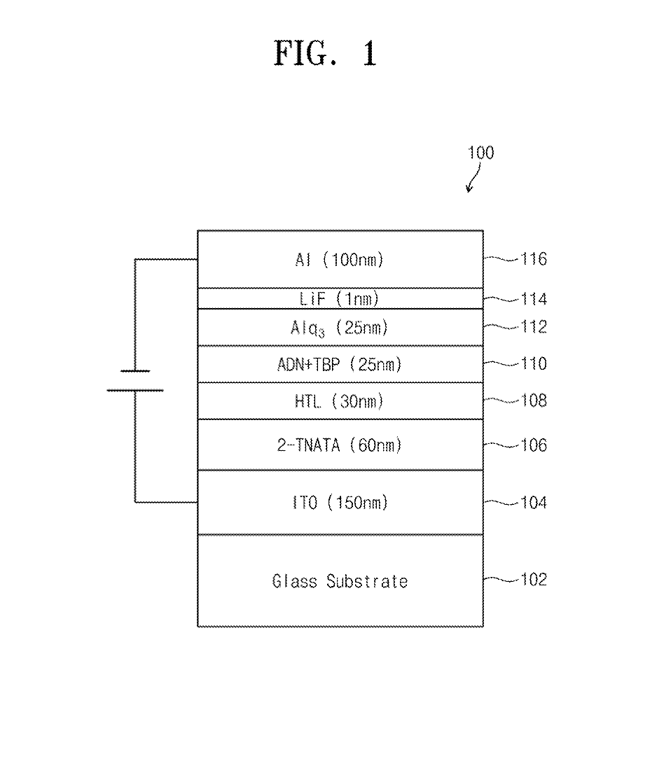

An organic EL device using the triarylamine derivative represented by Formula 3 according to an example embodiment will be now explained in connection with FIG. 1.

FIG. 1 illustrates a schematic diagram of an organic EL device 100 according to an example embodiment.

Referring to FIG. 1, the organic EL device 100 may include, for example, a substrate 102, a positive electrode 104, a hole injection layer 106, a hole transport layer 108, an emission layer 110, an electron transport layer 112, an electron injection layer 114, and a negative electrode 116. In an example embodiment, the triarylamine derivative represented by Formula 3 may be used in the emission layer of the organic EL device. In an example embodiment, the triarylamine derivative represented by Formula 3 may be used in a layer disposed between the emission layer and the positive electrode.

For example, an organic EL device using the material according to an example embodiment in the hole transport layer 108 will be explained. The substrate 102 may be, for example, a transparent glass substrate, a semiconductor substrate formed by using silicon, a flexible substrate of a resin, etc. The positive electrode 104 may be disposed on the substrate 102 and may be formed, for example, using indium tin oxide (ITO), indium zinc oxide (IZO), etc. The hole injection layer 106 may be disposed on the positive electrode 104 and may include, for example, 4,4′,4″-tris[2-naphthyl(phenyl)amino]triphenylamine(2-TNATA), N,N,N′,N′-tetrakis(3-methylphenyl)-3,3′-dimethylbenzidine (HMTPD), etc. The hole transport layer 108 may be disposed on the hole injection layer 106 and may be formed, for example, using the triarylamine derivative represented by Formula 3 according to an example embodiment. The emission layer 110 may be disposed on the hole transport layer 108 and may be formed, for example, using a host material including 9,10-di(2-naphthyl)anthracene (AND) doped with 2,5,8,11-tetra-tert-butylperylene (TBP), etc. The electron transport layer 112 may be disposed on the emission layer 110 and may be formed, for example, using a material that includes tris(8-hydroxyquinolinato)aluminum (Alq3). The electron injection layer 114 may be disposed on the electron transport layer 112 and may be formed, for example, using a material that includes lithium fluoride (LiF). The negative electrode 116 may be disposed on the electron injection layer 114 and may be formed, for example, using a metal such as Al, or a transparent material such as ITO, IZO, etc. The thin films may be formed by selecting an appropriate film forming method such as vacuum deposition, sputtering, various coatings, etc. according to the material used.

In the organic EL device 100 according to the present example embodiment, a hole transporting layer formed using the triarylamine derivative represented by Formula 3 according an embodiment may help realize driving at a low voltage and high efficiency. In addition, the triarylamine derivative represented by Formula 3 according to an embodiment may be applied in an organic EL display of an active matrix using a TFT.

Further, in the organic EL device 100 according to an example embodiment, using the triarylamine derivative represented by Formula 3 according to an embodiment in an emission layer, or in one layer of stacking layers disposed between the emission layer and the positive electrode, may help realize driving at a low voltage, high efficiency, and long life of the device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Example 1

### Preparation Method

The material for an organic EL device according to an embodiment may be synthesized, for example, as follows.

(Synthesis of Compound 1)

In a three-necked, 100 mL flask, 1.00 g of Compound A, 2.13 g of Compound B, 0.13 g of bis(dibenzylideneacetone)palladium(0) (Pd(dba)₂), 0.18 g of tri-tert-butylphosphine ((t-Bu)₃P), and 0.65 g of sodium tert-butoxide were added under an argon atmosphere, and heated and refluxed in a 30 mL of toluene solvent for 6 hours. After completing the reaction, the reactant was cooled in the air, and water was added. An organic layer was separated, and solvents were removed by distillation. The crude product thus obtained was separated by silica gel column chromatography (using a mixture solvent of dichloromethane and hexane), and then recrystallized using a mixture solvent of toluene and hexane to produce 2.65 g (yield 85%) of Compound 1 as a white solid.

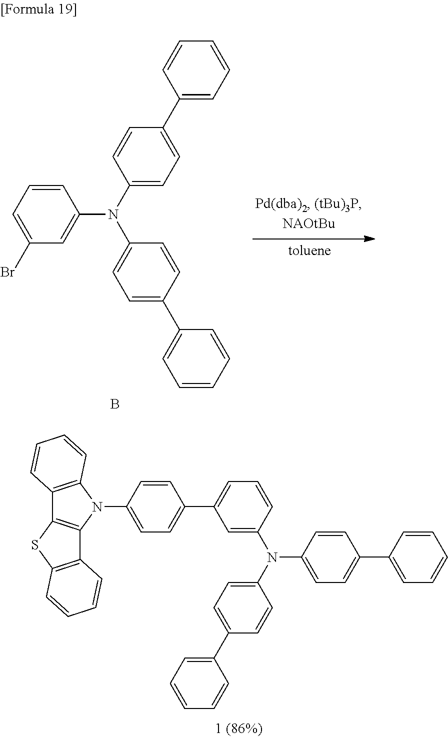

(Identification of Compound 1)

The chemical shift values of Compound 1 measured by ¹H-NMR were δ7.90 (d, 1H), 7.86-7.83 (m, 2H), 7.75-7.58 (m, 12H), 7.50-7.39 (m, 5H), and 7.35-7.22 (m, 14H). In addition, the molecular weight of Compound 1 measured by FAB-MS was 694.

(Synthesis of Compound 15 in Formula 20)

In a three-necked, 100 mL flask, 1.50 g of Compound A, 2.68 g of Compound C, 0.19 g of bis(dibenzylideneacetone)palladium(0) (Pd(dba)₂), 0.27 g of tri-tert-butylphosphine ((t-Bu)₃P) and 0.97 g of sodium tert-butoxide were added under an argon atmosphere, and heated and refluxed in a 45 mL of toluene solvent for 7 hours. After completing the reaction, the reactant was cooled in the air, and water was added. An organic layer was separated, and solvents were removed by distillation. The crude product thus obtained was separated by silica gel column chromatography (using a mixture solvent of dichloromethane and hexane), and then recrystallized using a mixture solvent of toluene and hexane to produce 3.31 g (yield 91%) of Compound 15 as a white solid.

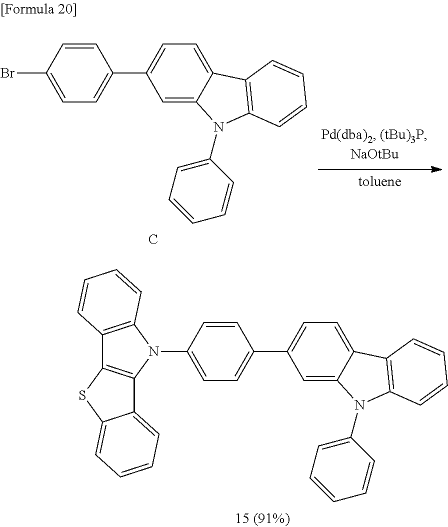

(Identification of Compound 15 in Formula 20)

The chemical shift values of Compound 15 measured by ¹H-NMR were δ8.14-8.12 (m, 2H), 7.95-7.77 (m, 4H), 7.72-7.50 (m, 8H), 7.46-7.38 (m, 5H), and 7.35-7.17 (m, 5H). The molecular weight of Compound 15 measured by FAB-MS was 540.

Organic EL devices were manufactured using Compounds 1, 15, and 33 (below) for Examples 1 to 3, respectively. In addition, organic EL devices were manufactured using Compounds C1 and C2 (below) for Comparative Examples 1 and 2, respectively. Compound C1 is a compound having an indolobenzopyrrole group and a carbazolyl group. Compound C2 is a compound having an indolobenzothiophenyl group and triarylamine combined via a single bond.

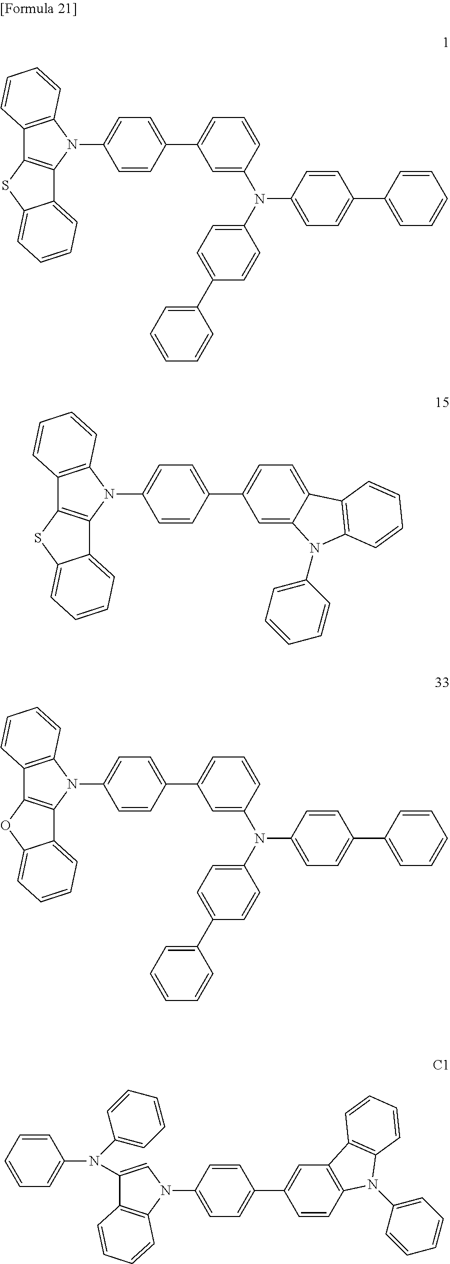 [SMILES: c1ccc(-c2ccc(N(c3ccc(-c4ccccc4)cc3)c3cccc(-c4ccc(-n5c6ccccc6c6sc7ccccc7c65)cc4)c3)cc2)cc1 ; c1ccc(-n2c3ccccc3c3ccc(-c4ccc(-n5c6ccccc6c6sc7ccccc7c65)cc4)cc32)cc1 ; c1ccc(-c2ccc(N(c3ccc(-c4ccccc4)cc3)c3cccc(-c4ccc(-n5c6ccccc6c6oc7ccccc7c65)cc4)c3)cc2)cc1 ; c1ccc(N(c2ccccc2)c2cn(-c3ccc(-c4ccc5c(c4)c4ccccc4n5-c4ccccc4)cc3)c3ccccc23)cc1]

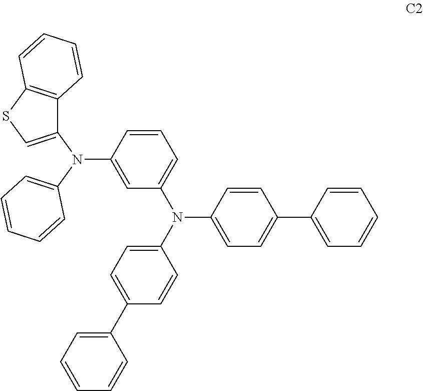 [SMILES: c1ccc(-c2ccc(N(c3ccc(-c4ccccc4)cc3)c3cccc(N(c4ccccc4)c4csc5ccccc45)c3)cc2)cc1]

Organic EL devices were manufactured by using the materials according to Examples 1 to 3 and Comparative Examples 1 and 2 as hole transporting materials by the above-described method. In detail, the substrate 102 was formed by using a transparent glass substrate, the positive electrode 104 was formed using ITO to a thickness of about 150 nm, the hole injection layer 106 was formed using TNATA to a thickness of about 60 nm, the hole transport layer 108 was formed to a thickness of about 30 nm, the emission layer 110 was formed by using ADN doped with 3% TBP to a thickness of about 25 nm, the electron transport layer 112 was formed using Alq3 to a thickness of about 25 nm, the electron injection layer 114 was formed by using LiF to a thickness of about 1 nm, and the negative electrode 116 was formed by using Al to a thickness of about 100 nm.

With respect to the organic EL devices thus manufactured, the voltage, the current efficiency, and half life were evaluated. The current efficiency means values at the current density of 10 mA/cm² and measured and evaluated at the half life of 1,000 cd/m².

TABLE 1

| | | Current efficiency | |
|---|---|---|---|
| | Voltage (V) | (Cd/A) | Life (hr) |
| Example 1 | 5.8 | 7.5 | 1,400 |
| Example 2 | 6.1 | 7.6 | 1,600 |
| Example 3 | 6.0 | 7.5 | 1,500 |
| Comparative Example 1 | 6.5 | 6.4 | 1,200 |
| Comparative Example 2 | 7.0 | 6.2 | 1,100 |

As shown in Table 1, the organic EL devices including the compounds according to Examples 1 to 3 were driven at a lower voltage when compared to those including the compounds according to Comparative Examples 1 and 2. In addition, the current efficiency and the half life were also better for the compounds according to Examples 1 to 3 when compared to those for the compounds according to Comparative Examples 1 and 2. When comparing the compound of Example 1 and the compound of Comparative Example 2, remarkable lowering of the driving voltage and the realization of high efficiency could be attained when using the compound of Example 1 including a phenylene group as a connecting group. When comparing the compound of Example 1 and the compound of Example 2, it could be confirmed that hole transporting properties were improved through the expansion of a conjugate system by forming a carbazolyl group via combining R₃ and R₄, thereby improving the emission efficiency.

In addition, when comparing the compound of Example 2 and the compound of Comparative Example 1, the inclusion of a phenylene group as a connecting group and the carbazole group were common. However, the compound of Example 2 including an indolobenzothiophenyl group realized the driving at a low voltage and high efficiency when compared to the compound of Comparative Example 1 including an indolobenzopyrrole group. These effects were also obtained for the compound of Example 3 including an indolobenzofuranyl group. As a result, significant effects were obtained when using a compound including the indolobenzothiophenyl group or the indolobenzofuranyl group relative to the indolobenzopyrrole group.

In the triarylamine derivative represented by Formula 3 according to an example embodiment, an indolobenzofuranyl group or an indolobenzothiophene group is used, and a triarylamine part is combined via a connecting group. Thus, hole transporting properties may be increased, and the driving at a low voltage, the high emission efficiency, and the long life of an organic EL device may be realized.

By way of summation and review, an example of an organic electroluminescence device (referred to as an organic EL device) is an organic EL device that includes a positive electrode, a hole transport layer disposed on the positive electrode, an emission layer disposed on the hole transport layer, an electron transport layer disposed on the emission layer, and a negative electrode disposed on the electron transport layer. Holes injected from the positive electrode are injected into the emission layer via the hole transport layer. Meanwhile, electrons are injected from the negative electrode, and then injected into the emission layer via the electron transport layer. The holes and the electrons injected into the emission layer are recombined to generate excitons within the emission layer. The organic EL device emits light by using light generated by the radiation and deactivation of the excitons. The organic EL device may have various configurations.

For applying an organic EL device in a display device, the organic EL device is desired to be driven at a low voltage and have long life. To realize the driving at the low voltage and the high efficiency of the organic EL device, the normalization and the stabilization of a hole transport layer have been examined. As a hole transport material used in the hole transport layer, various compounds such as a carbazole derivative and an aromatic amine-based compound may be considered.

An indolobenzothiophene or indoloindole derivative has been studied as a material of a host material for realizing the high efficiency or the long life of a device. However, an organic EL device manufactured using those materials may not have high emission efficiency. Also, the emission efficiency of an organic EL device in a blue emission region may be lower than that in a red emission region or in a green emission region, and so the improvement of the emission efficiency in the blue emission region is desired.

As described above, embodiments relate to a material for an organic electroluminescence device that may be driven at a low voltage and have high efficiency, and an organic electroluminescence device using the same. Embodiments may provide a material for an organic EL device capable of being driven at a low voltage, and having high efficiency and long life, and an organic EL device using the same.

According to an example embodiment, combining an indolobenzofuran part or an indolobenzothiophene part with a triarylamine part may lower a driving voltage, which may help realize the high efficiency and the long life of an organic EL device. A lower highest occupied molecular orbital (HOMO) level may be desired when the material is used as a material of an emission layer or a stacking layer disposed between the emission layer and a positive electrode. According to an example embodiment, introducing an indolobenzofuran part or an indolobenzothiophene part, instead of, for example, an indolobenzopyrrole part, may provide a significant lowering of driving voltage, and may help realize high efficiency and long life. Also, the control of the HOMO level of the material of an organic EL device may be efficient when the indolobenzofuran part or the indolobenzothiophene part are combined with the triarylamine part using a connecting group, instead of, for example, a single bond.

According to an example embodiment, a material for the organic EL device may exhibit improved hole transporting properties by condensing benzothiophene or benzofuran with respect to indole exhibiting hole transporting properties, and may exhibit a controlled highest occupied molecular orbital (HOMO) level. Thus, an organic EL device driven at a low voltage and having high efficiency and long life may be manufactured.

According to an example embodiment, an organic EL device may realize driving at a low voltage and high efficiency by including the material for the organic EL device having triarylamine with an indolobenzofuran part or an indolobenzothiophene part in the emission layer.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims

What is claimed is:

1. A material for an organic electroluminescence (EL) device, the material comprising a triarylamine derivative represented by the following Formula 1: 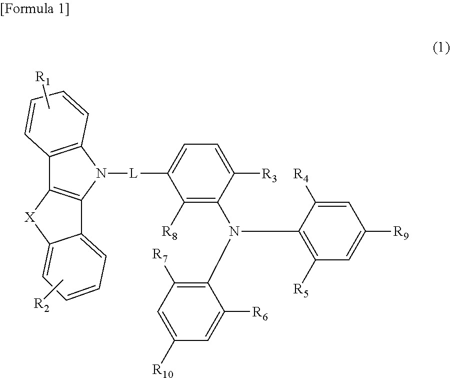
wherein:
X is an oxygen atom or a sulfur atom,
R₁ and R₂ are independently a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom,
R₃ to R₁₀ are independently a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, oxygen, sulfur, or a bond to another one of R₃ to R₁₀, and
L is an arylene group.

2. The material as claimed in claim 1, wherein two or more of R₃ to R₈ in Formula 1 are combined to each other to form a saturated or unsaturated ring.

3. The material as claimed in claim 2, wherein R₃ and R₄ in Formula 1 are combined to each other to form a chemical structure represented by the following Formula 2: 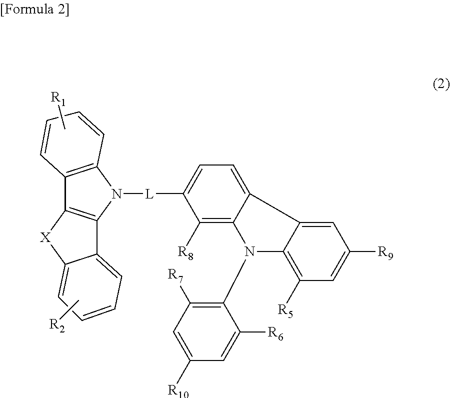
wherein:
X is an oxygen atom or a sulfur atom,
R₁ and R₂ are independently a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom,
R₅ to R₁₀ are independently a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom, and
L is an arylene group.

4. The material as claimed in claim 2, wherein two or more of R₅ to R₈ in Formula 1 are combined to each other to form a saturated or unsaturated ring.

5. An organic electroluminescence (EL) device comprising a material for an organic EL device including a triarylamine derivative represented by the following Formula 3, the triarylamine derivative represented by Formula 3 being in at least one layer of an emission layer and a stacking layer disposed between an emission layer and a positive electrode: 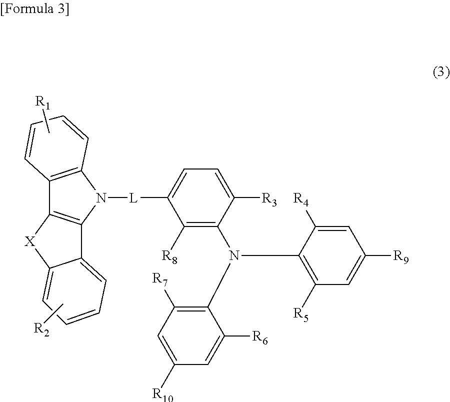
wherein:
X is an oxygen atom or a sulfur atom,
R₁ and R₂ are independently a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom,
R₃ to R₁₀ are independently a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, oxygen, sulfur, or a bond to another one of R₃ to R₁₀, and
L is an arylene group.

6. The device as claimed in claim 5, wherein two or more of R₃ to R₈ in Formula 3 are combined to each other to form a saturated or unsaturated ring.

7. The device as claimed in claim 6, wherein R₃ and R₄ in Formula 3 are combined to each other to form a chemical structure represented by the following Formula 4: 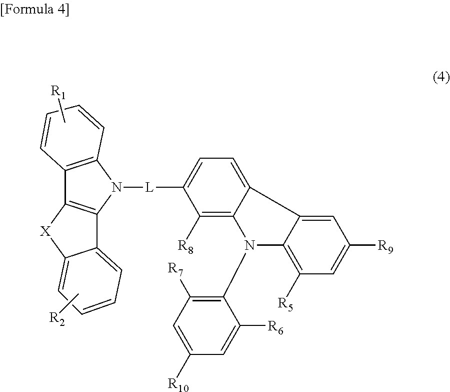
wherein:
X is an oxygen atom or a sulfur atom,
R₁ and R₂ are independently a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom,
R₅ to R₁₀ are independently a hydrogen atom, a deuterium atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, or a halogen atom, and
L is an arylene group.

8. The device as claimed in claim 6, wherein two or more of R₅ to R₈ in Formula 3 are combined to each other to form a saturated or unsaturated ring.

9. The device as claimed in claim 5, wherein the triarylamine derivative represented by Formula 3 is selected from compounds in the following Formula 5:

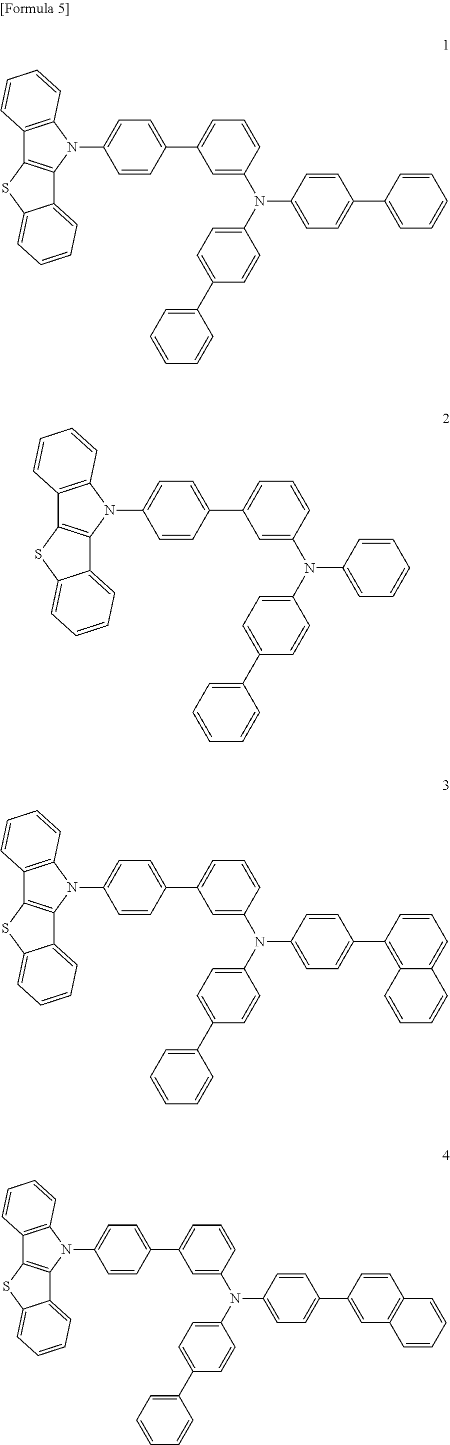 [SMILES: c1ccc(-c2ccc(N(c3ccc(-c4ccccc4)cc3)c3cccc(-c4ccc(-n5c6ccccc6c6sc7ccccc7c65)cc4)c3)cc2)cc1 ; c1ccc(-c2ccc(N(c3ccccc3)c3cccc(-c4ccc(-n5c6ccccc6c6sc7ccccc7c65)cc4)c3)cc2)cc1 ; c1ccc(-c2ccc(N(c3ccc(-c4cccc5ccccc45)cc3)c3cccc(-c4ccc(-n5c6ccccc6c6sc7ccccc7c65)cc4)c3)cc2)cc1 ; c1ccc(-c2ccc(N(c3ccc(-c4ccc5ccccc5c4)cc3)c3cccc(-c4ccc(-n5c6ccccc6c6sc7ccccc7c65)cc4)c3)cc2)cc1]

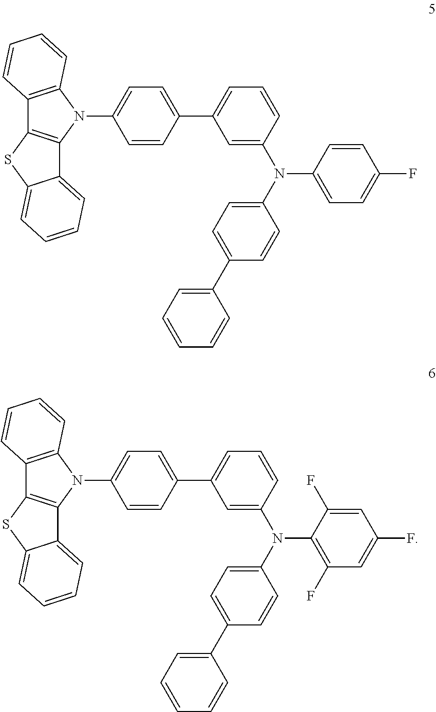 [SMILES: Fc1ccc(N(c2ccc(-c3ccccc3)cc2)c2cccc(-c3ccc(-n4c5ccccc5c5sc6ccccc6c54)cc3)c2)cc1 ; Fc1cc(F)c(N(c2ccc(-c3ccccc3)cc2)c2cccc(-c3ccc(-n4c5ccccc5c5sc6ccccc6c54)cc3)c2)c(F)c1]

10. The device as claimed in claim 5, wherein the triarylamine derivative represented by Formula 3 is selected from compounds in the following Formula 6:

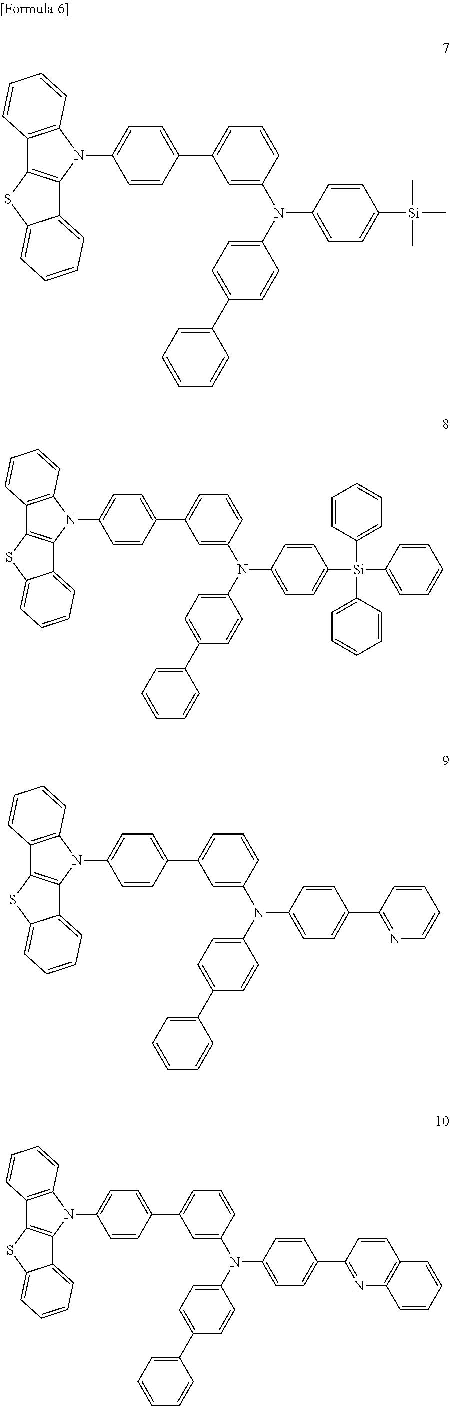 [SMILES: C[Si](C)(C)c1ccc(N(c2ccc(-c3ccccc3)cc2)c2cccc(-c3ccc(-n4c5ccccc5c5sc6ccccc6c54)cc3)c2)cc1 ; c1ccc(-c2ccc(N(c3ccc([Si](c4ccccc4)(c4ccccc4)c4ccccc4)cc3)c3cccc(-c4ccc(-n5c6ccccc6c6sc7ccccc7c65)cc4)c3)cc2)cc1 ; c1ccc(-c2ccc(N(c3ccc(-c4ccccn4)cc3)c3cccc(-c4ccc(-n5c6ccccc6c6sc7ccccc7c65)cc4)c3)cc2)cc1 ; c1ccc(-c2ccc(N(c3ccc(-c4ccc5ccccc5n4)cc3)c3cccc(-c4ccc(-n5c6ccccc6c6sc7ccccc7c65)cc4)c3)cc2)cc1]

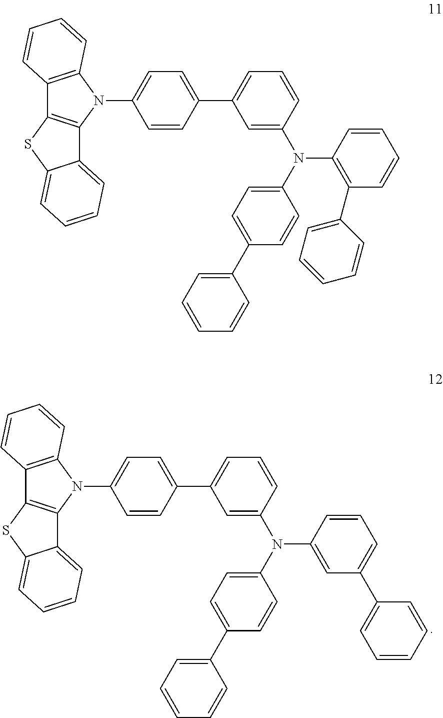 [SMILES: c1ccc(-c2ccc(N(c3cccc(-c4ccc(-n5c6ccccc6c6sc7ccccc7c65)cc4)c3)c3ccccc3-c3ccccc3)cc2)cc1 ; c1ccc(-c2ccc(N(c3cccc(-c4ccccc4)c3)c3cccc(-c4ccc(-n5c6ccccc6c6sc7ccccc7c65)cc4)c3)cc2)cc1]

11. The device as claimed in claim 5, wherein the triarylamine derivative represented by Formula 3 is selected from compounds in the following Formula 7:

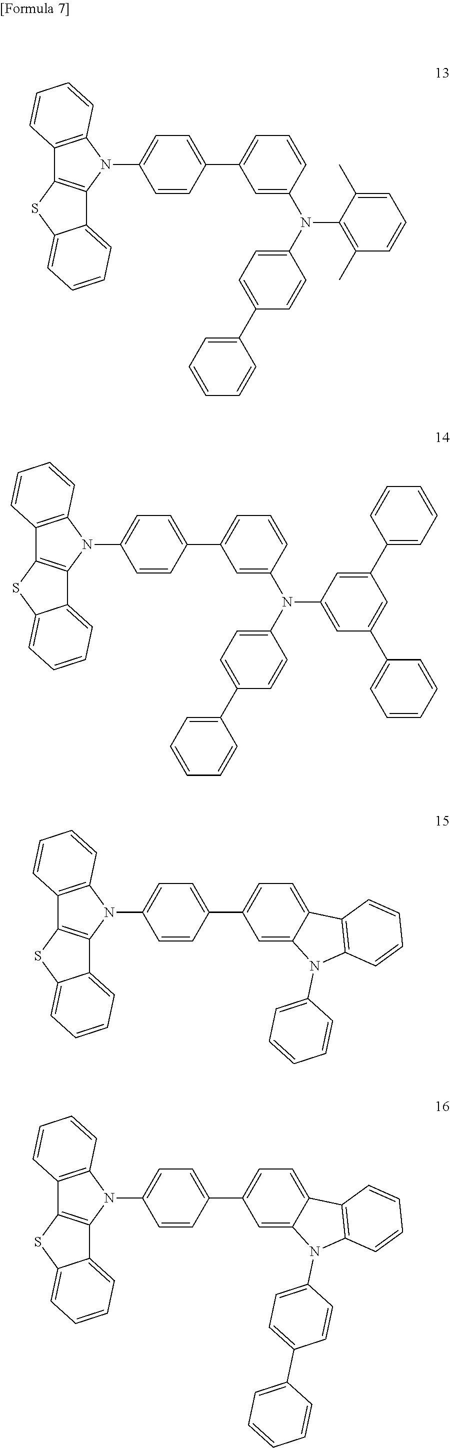 [SMILES: Cc1cccc(C)c1N(c1ccc(-c2ccccc2)cc1)c1cccc(-c2ccc(-n3c4ccccc4c4sc5ccccc5c43)cc2)c1 ; c1ccc(-c2ccc(N(c3cccc(-c4ccc(-n5c6ccccc6c6sc7ccccc7c65)cc4)c3)c3cc(-c4ccccc4)cc(-c4ccccc4)c3)cc2)cc1 ; c1ccc(-n2c3ccccc3c3ccc(-c4ccc(-n5c6ccccc6c6sc7ccccc7c65)cc4)cc32)cc1 ; c1ccc(-c2ccc(-n3c4ccccc4c4ccc(-c5ccc(-n6c7ccccc7c7sc8ccccc8c76)cc5)cc43)cc2)cc1]

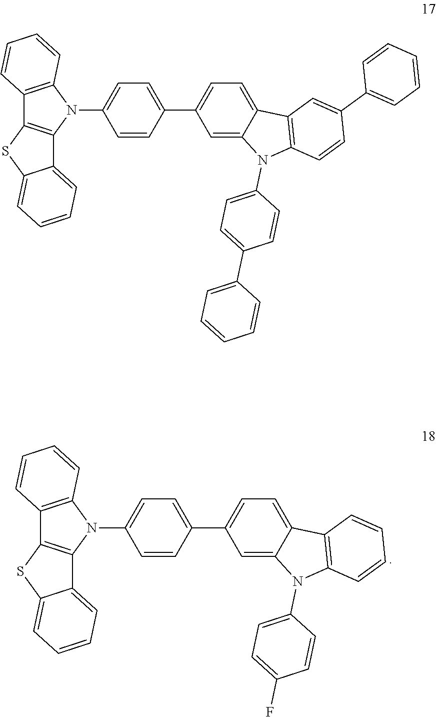 [SMILES: c1ccc(-c2ccc(-n3c4ccc(-c5ccccc5)cc4c4ccc(-c5ccc(-n6c7ccccc7c7sc8ccccc8c76)cc5)cc43)cc2)cc1 ; Fc1ccc(-n2c3ccccc3c3ccc(-c4ccc(-n5c6ccccc6c6sc7ccccc7c65)cc4)cc32)cc1]

12. The device as claimed in claim 5, wherein the triarylamine derivative represented by Formula 3 is selected from compounds in the following Formula 8:

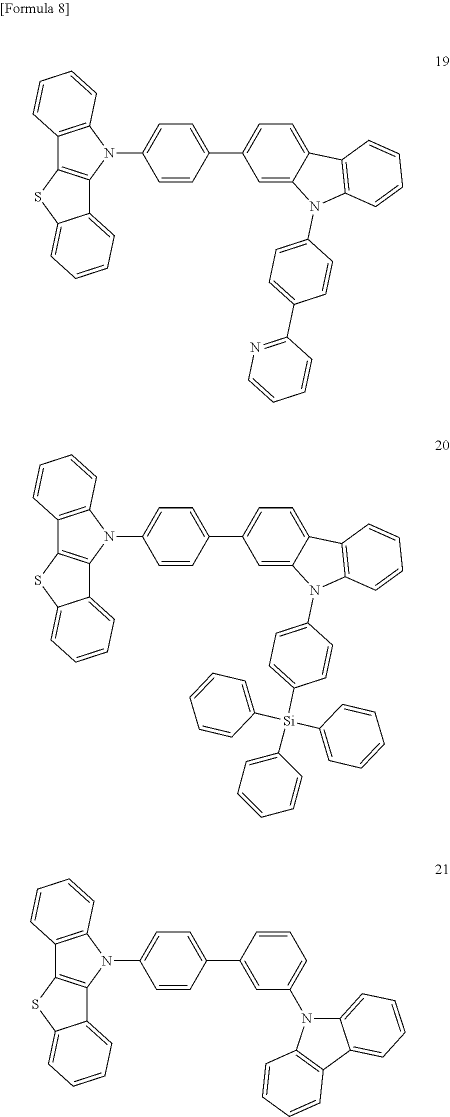 [SMILES: c1ccc(-c2ccc(-n3c4ccccc4c4ccc(-c5ccc(-n6c7ccccc7c7sc8ccccc8c76)cc5)cc43)cc2)nc1 ; c1ccc([Si](c2ccccc2)(c2ccccc2)c2ccc(-n3c4ccccc4c4ccc(-c5ccc(-n6c7ccccc7c7sc8ccccc8c76)cc5)cc43)cc2)cc1 ; c1cc(-c2ccc(-n3c4ccccc4c4sc5ccccc5c43)cc2)cc(-n2c3ccccc3c3ccccc32)c1]

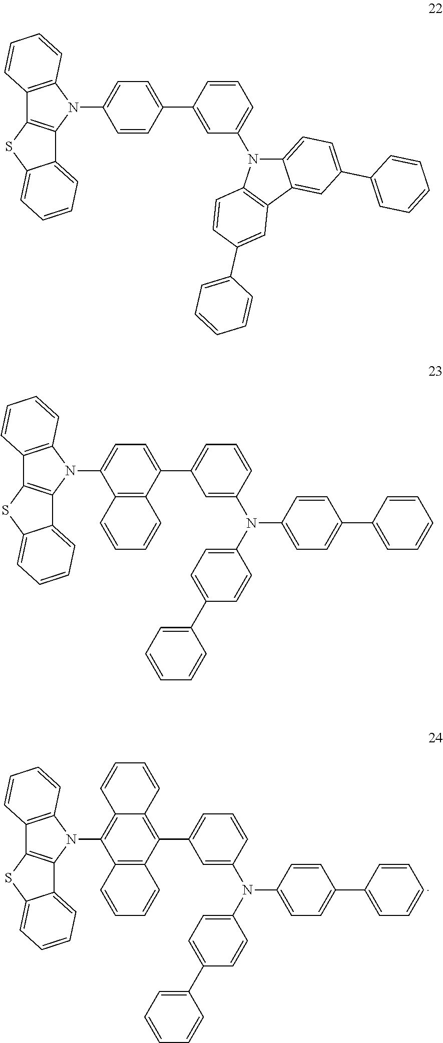 [SMILES: c1ccc(-c2ccc3c(c2)c2cc(-c4ccccc4)ccc2n3-c2cccc(-c3ccc(-n4c5ccccc5c5sc6ccccc6c54)cc3)c2)cc1 ; c1ccc(-c2ccc(N(c3ccc(-c4ccccc4)cc3)c3cccc(-c4ccc(-n5c6ccccc6c6sc7ccccc7c65)c5ccccc45)c3)cc2)cc1 ; c1ccc(-c2ccc(N(c3ccc(-c4ccccc4)cc3)c3cccc(-c4c5ccccc5c(-n5c6ccccc6c6sc7ccccc7c65)c5ccccc45)c3)cc2)cc1]

13. The device as claimed in claim 5, wherein the triarylamine derivative represented by Formula 3 is selected from compounds in the following Formula 9:

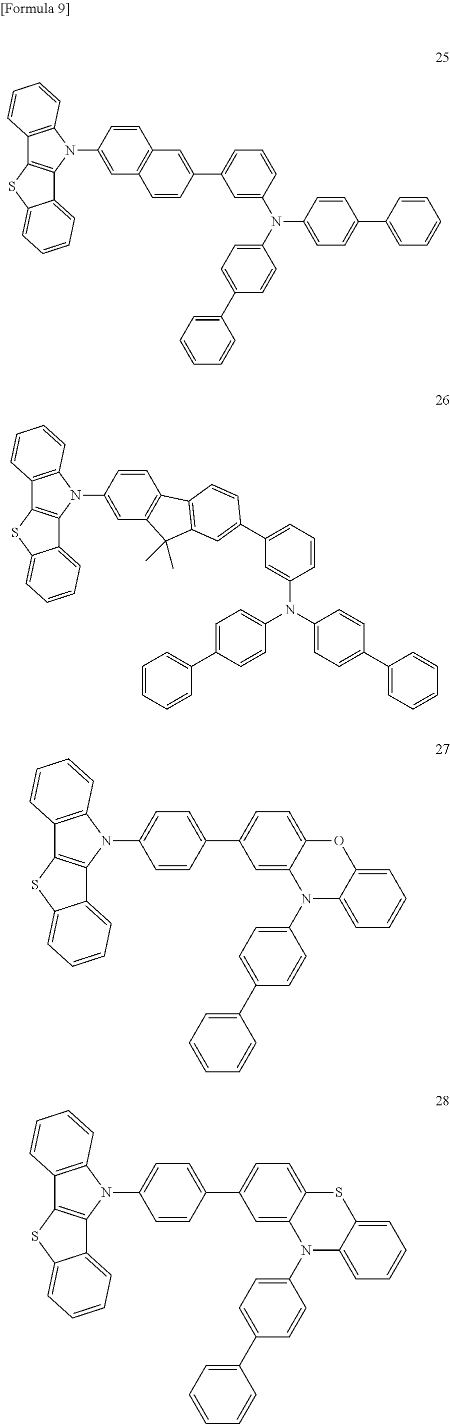 [SMILES: c1ccc(-c2ccc(N(c3ccc(-c4ccccc4)cc3)c3cccc(-c4ccc5cc(-n6c7ccccc7c7sc8ccccc8c76)ccc5c4)c3)cc2)cc1 ; CC1(C)c2cc(-c3cccc(N(c4ccc(-c5ccccc5)cc4)c4ccc(-c5ccccc5)cc4)c3)ccc2-c2ccc(-n3c4ccccc4c4sc5ccccc5c43)cc21 ; c1ccc(-c2ccc(N3c4ccccc4Oc4ccc(-c5ccc(-n6c7ccccc7c7sc8ccccc8c76)cc5)cc43)cc2)cc1 ; c1ccc(-c2ccc(N3c4ccccc4Sc4ccc(-c5ccc(-n6c7ccccc7c7sc8ccccc8c76)cc5)cc43)cc2)cc1]

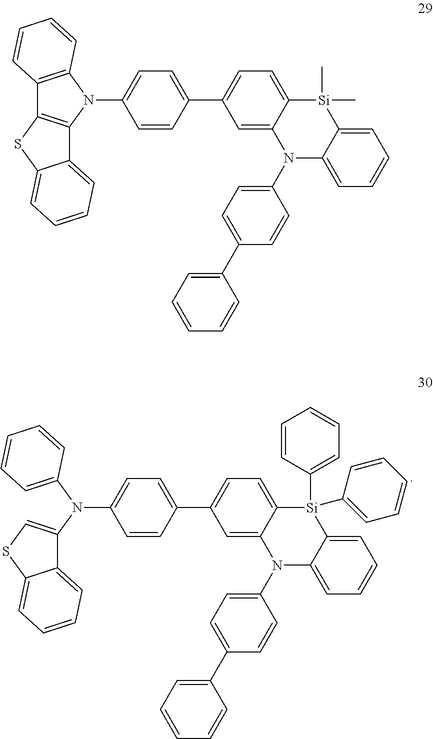 [SMILES: C[Si]1(C)c2ccccc2N(c2ccc(-c3ccccc3)cc2)c2cc(-c3ccc(-n4c5ccccc5c5sc6ccccc6c54)cc3)ccc21 ; c1ccc(-c2ccc(N3c4ccccc4[Si](c4ccccc4)(c4ccccc4)c4ccc(-c5ccc(N(c6ccccc6)c6csc7ccccc67)cc5)cc43)cc2)cc1]

14. The device as claimed in claim 5, wherein the triarylamine derivative represented by Formula 3 is selected from compounds in the following Formula 10:

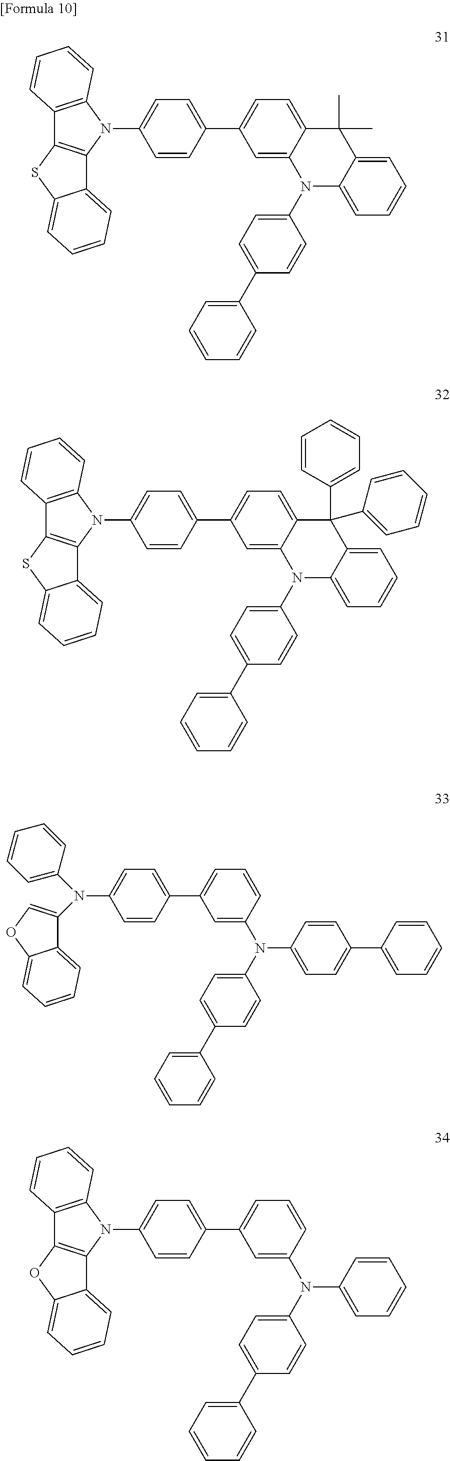 [SMILES: CC1(C)c2ccccc2N(c2ccc(-c3ccccc3)cc2)c2cc(-c3ccc(-n4c5ccccc5c5sc6ccccc6c54)cc3)ccc21 ; c1ccc(-c2ccc(N3c4ccccc4C(c4ccccc4)(c4ccccc4)c4ccc(-c5ccc(-n6c7ccccc7c7sc8ccccc8c76)cc5)cc43)cc2)cc1 ; c1ccc(-c2ccc(N(c3ccc(-c4ccccc4)cc3)c3cccc(-c4ccc(N(c5ccccc5)c5coc6ccccc56)cc4)c3)cc2)cc1 ; c1ccc(-c2ccc(N(c3ccccc3)c3cccc(-c4ccc(-n5c6ccccc6c6oc7ccccc7c65)cc4)c3)cc2)cc1]

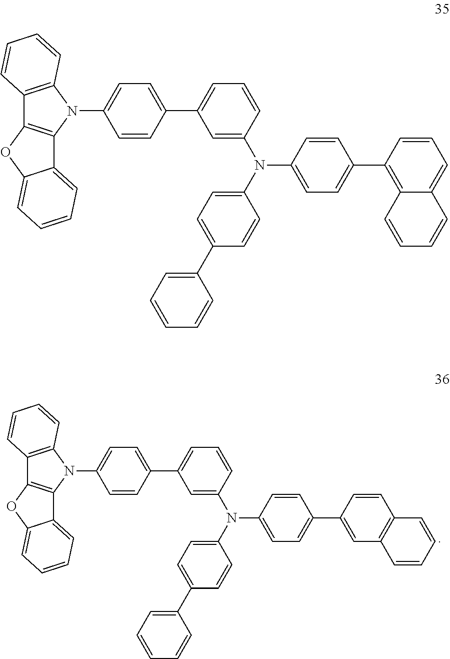 [SMILES: c1ccc(-c2ccc(N(c3ccc(-c4cccc5ccccc45)cc3)c3cccc(-c4ccc(-n5c6ccccc6c6oc7ccccc7c65)cc4)c3)cc2)cc1 ; c1ccc(-c2ccc(N(c3ccc(-c4ccc5ccccc5c4)cc3)c3cccc(-c4ccc(-n5c6ccccc6c6oc7ccccc7c65)cc4)c3)cc2)cc1]

15. The device as claimed in claim 5, wherein the triarylamine derivative represented by Formula 3 is selected from compounds in the following Formula 11:

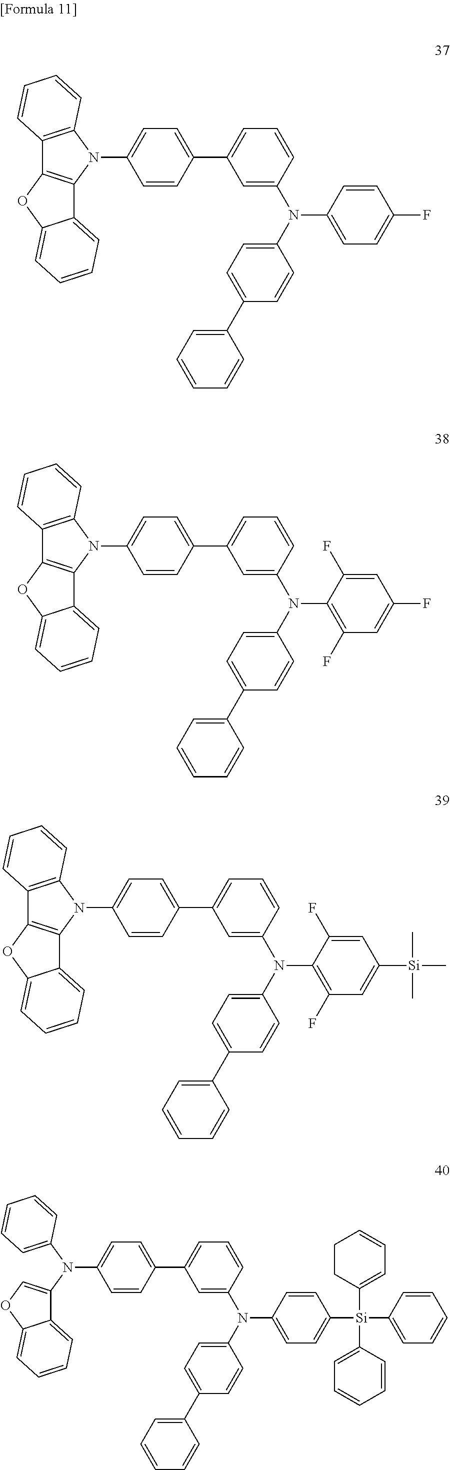 [SMILES: Fc1ccc(N(c2ccc(-c3ccccc3)cc2)c2cccc(-c3ccc(-n4c5ccccc5c5oc6ccccc6c54)cc3)c2)cc1 ; Fc1cc(F)c(N(c2ccc(-c3ccccc3)cc2)c2cccc(-c3ccc(-n4c5ccccc5c5oc6ccccc6c54)cc3)c2)c(F)c1 ; C[Si](C)(C)c1cc(F)c(N(c2ccc(-c3ccccc3)cc2)c2cccc(-c3ccc(-n4c5ccccc5c5oc6ccccc6c54)cc3)c2)c(F)c1 ; C1=CCCC([Si](c2ccccc2)(c2ccccc2)c2ccc(N(c3ccc(-c4ccccc4)cc3)c3cccc(-c4ccc(N(c5ccccc5)c5coc6ccccc56)cc4)c3)cc2)=C1]

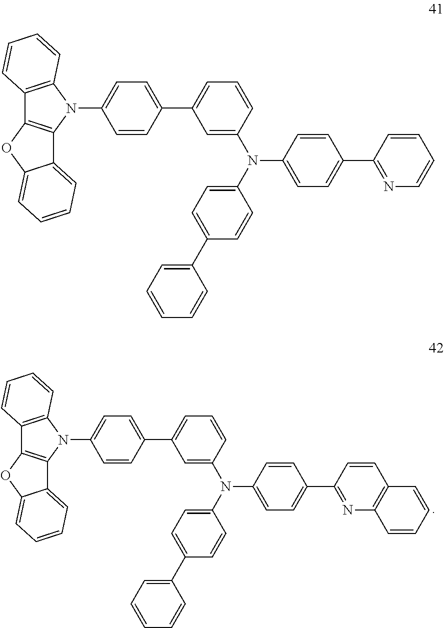 [SMILES: c1ccc(-c2ccc(N(c3ccc(-c4ccccn4)cc3)c3cccc(-c4ccc(-n5c6ccccc6c6oc7ccccc7c65)cc4)c3)cc2)cc1 ; c1ccc(-c2ccc(N(c3ccc(-c4ccc5ccccc5n4)cc3)c3cccc(-c4ccc(-n5c6ccccc6c6oc7ccccc7c65)cc4)c3)cc2)cc1]

16. The device as claimed in claim 5, wherein the triarylamine derivative represented by Formula 3 is selected from compounds in the following Formula 12:

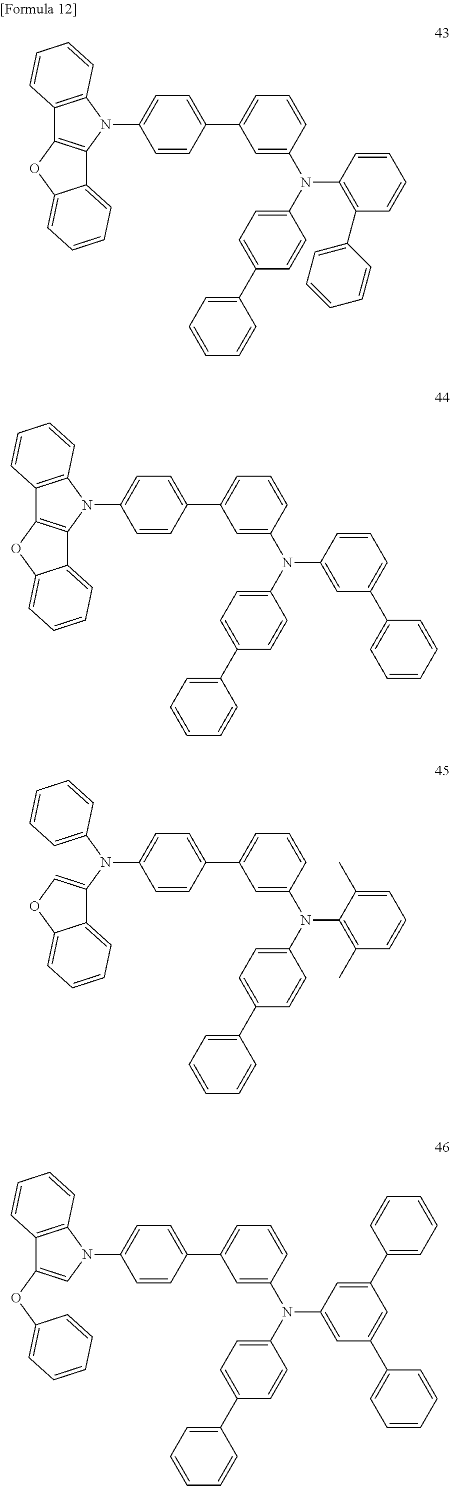 [SMILES: c1ccc(-c2ccc(N(c3cccc(-c4ccc(-n5c6ccccc6c6oc7ccccc7c65)cc4)c3)c3ccccc3-c3ccccc3)cc2)cc1 ; c1ccc(-c2ccc(N(c3cccc(-c4ccccc4)c3)c3cccc(-c4ccc(-n5c6ccccc6c6oc7ccccc7c65)cc4)c3)cc2)cc1 ; Cc1cccc(C)c1N(c1ccc(-c2ccccc2)cc1)c1cccc(-c2ccc(N(c3ccccc3)c3coc4ccccc34)cc2)c1 ; c1ccc(Oc2cn(-c3ccc(-c4cccc(N(c5ccc(-c6ccccc6)cc5)c5cc(-c6ccccc6)cc(-c6ccccc6)c5)c4)cc3)c3ccccc23)cc1]

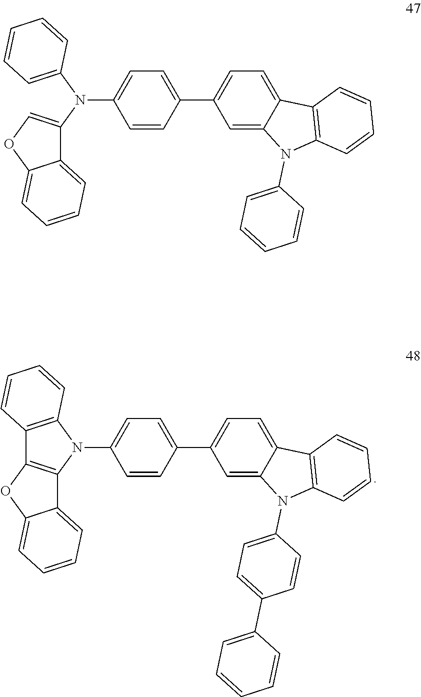 [SMILES: c1ccc(N(c2ccc(-c3ccc4c5ccccc5n(-c5ccccc5)c4c3)cc2)c2coc3ccccc23)cc1 ; c1ccc(-c2ccc(-n3c4ccccc4c4ccc(-c5ccc(-n6c7ccccc7c7oc8ccccc8c76)cc5)cc43)cc2)cc1]

17. The device as claimed in claim 5, wherein the triarylamine derivative represented by Formula 3 is selected from compounds in the following Formula 13:

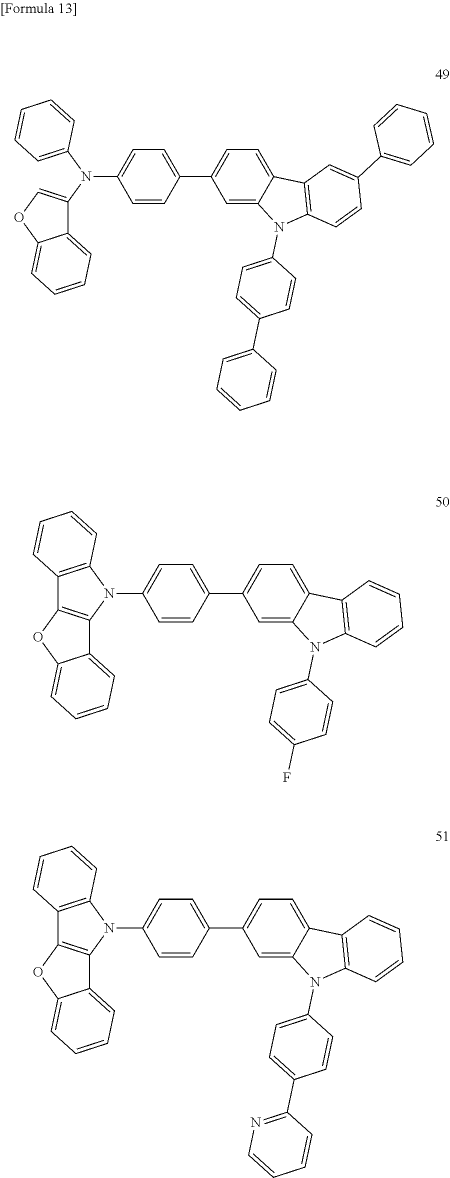 [SMILES: c1ccc(-c2ccc(-n3c4ccc(-c5ccccc5)cc4c4ccc(-c5ccc(N(c6ccccc6)c6coc7ccccc67)cc5)cc43)cc2)cc1 ; Fc1ccc(-n2c3ccccc3c3ccc(-c4ccc(-n5c6ccccc6c6oc7ccccc7c65)cc4)cc32)cc1 ; c1ccc(-c2ccc(-n3c4ccccc4c4ccc(-c5ccc(-n6c7ccccc7c7oc8ccccc8c76)cc5)cc43)cc2)nc1]

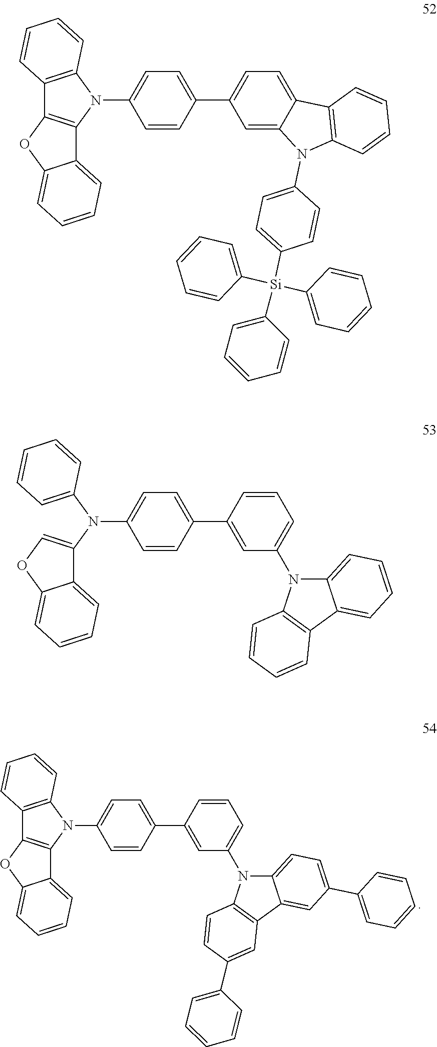 [SMILES: c1ccc([Si](c2ccccc2)(c2ccccc2)c2ccc(-n3c4ccccc4c4ccc(-c5ccc(-n6c7ccccc7c7oc8ccccc8c76)cc5)cc43)cc2)cc1 ; c1ccc(N(c2ccc(-c3cccc(-n4c5ccccc5c5ccccc54)c3)cc2)c2coc3ccccc23)cc1 ; c1ccc(-c2ccc3c(c2)c2cc(-c4ccccc4)ccc2n3-c2cccc(-c3ccc(-n4c5ccccc5c5oc6ccccc6c54)cc3)c2)cc1]

18. The device as claimed in claim 5, wherein the triarylamine derivative represented by Formula 3 is selected from compounds in the following Formulae 14 and 15:

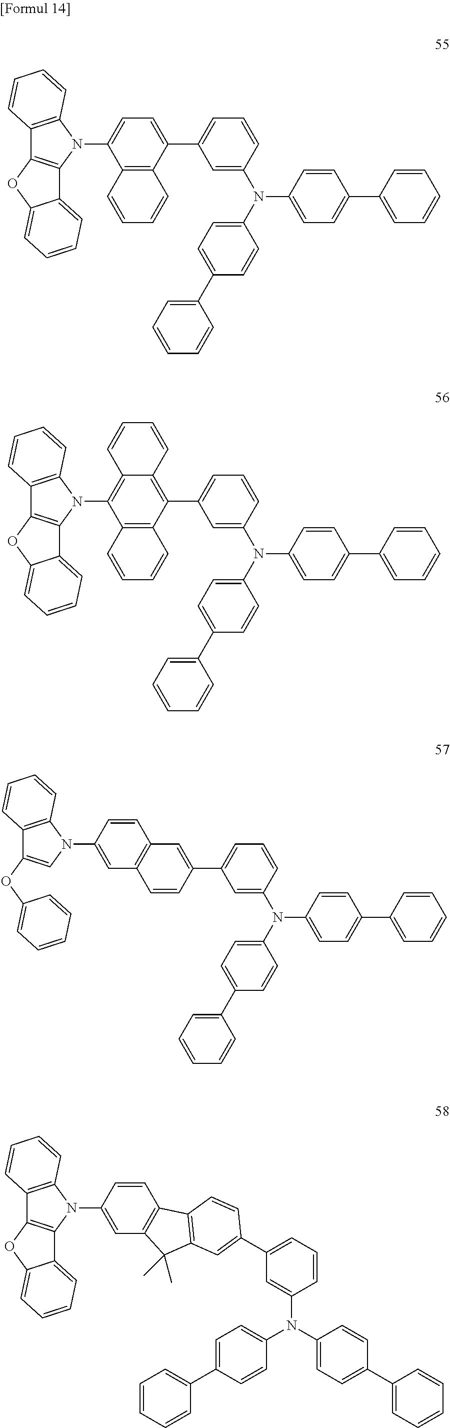 [SMILES: c1ccc(-c2ccc(N(c3ccc(-c4ccccc4)cc3)c3cccc(-c4ccc(-n5c6ccccc6c6oc7ccccc7c65)c5ccccc45)c3)cc2)cc1 ; c1ccc(-c2ccc(N(c3ccc(-c4ccccc4)cc3)c3cccc(-c4c5ccccc5c(-n5c6ccccc6c6oc7ccccc7c65)c5ccccc45)c3)cc2)cc1 ; c1ccc(Oc2cn(-c3ccc4cc(-c5cccc(N(c6ccc(-c7ccccc7)cc6)c6ccc(-c7ccccc7)cc6)c5)ccc4c3)c3ccccc23)cc1 ; CC1(C)c2cc(-c3cccc(N(c4ccc(-c5ccccc5)cc4)c4ccc(-c5ccccc5)cc4)c3)ccc2-c2ccc(-n3c4ccccc4c4oc5ccccc5c43)cc21]

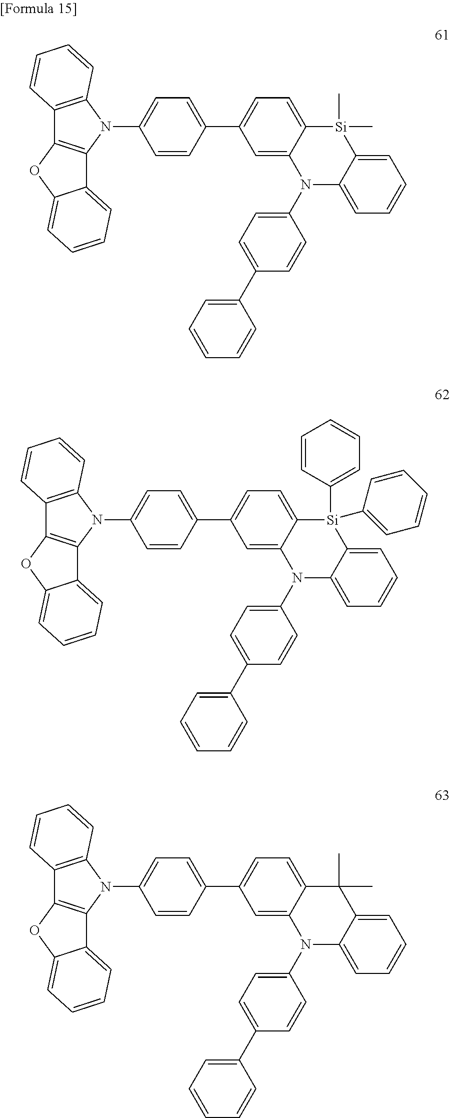 [SMILES: C[Si]1(C)c2ccccc2N(c2ccc(-c3ccccc3)cc2)c2cc(-c3ccc(-n4c5ccccc5c5oc6ccccc6c54)cc3)ccc21 ; c1ccc(-c2ccc(N3c4ccccc4[Si](c4ccccc4)(c4ccccc4)c4ccc(-c5ccc(-n6c7ccccc7c7oc8ccccc8c76)cc5)cc43)cc2)cc1 ; CC1(C)c2ccccc2N(c2ccc(-c3ccccc3)cc2)c2cc(-c3ccc(-n4c5ccccc5c5oc6ccccc6c54)cc3)ccc21]

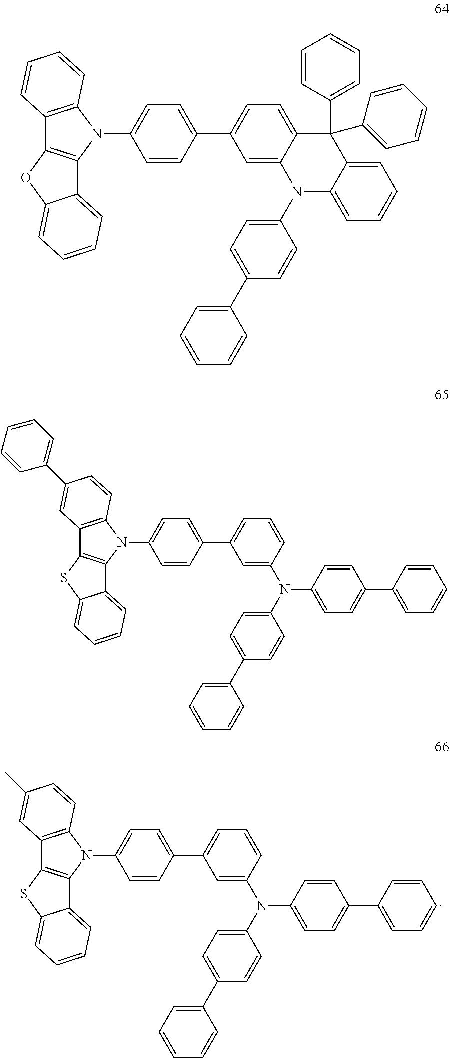 [SMILES: c1ccc(-c2ccc(N3c4ccccc4C(c4ccccc4)(c4ccccc4)c4ccc(-c5ccc(-n6c7ccccc7c7oc8ccccc8c76)cc5)cc43)cc2)cc1 ; c1ccc(-c2ccc(N(c3ccc(-c4ccccc4)cc3)c3cccc(-c4ccc(-n5c6ccc(-c7ccccc7)cc6c6sc7ccccc7c65)cc4)c3)cc2)cc1 ; Cc1ccc2c(c1)c1sc3ccccc3c1n2-c1ccc(-c2cccc(N(c3ccc(-c4ccccc4)cc3)c3ccc(-c4ccccc4)cc3)c2)cc1]

19. The device as claimed in claim 5, wherein the triarylamine derivative represented by Formula 3 is selected from compounds in the following Formulae 16, 17, and 18: 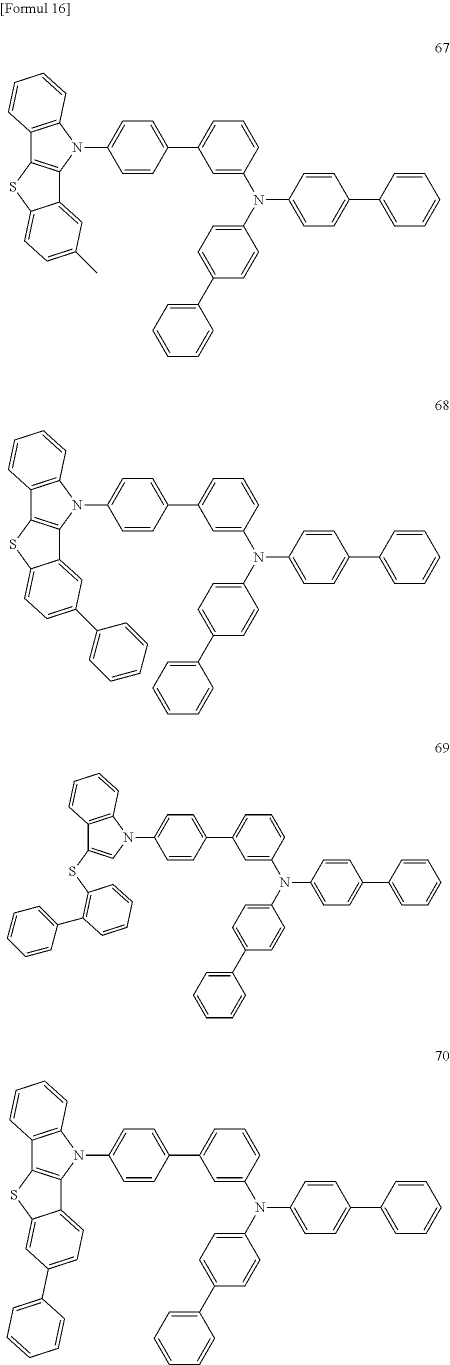 [SMILES: Cc1ccc2sc3c4ccccc4n(-c4ccc(-c5cccc(N(c6ccc(-c7ccccc7)cc6)c6ccc(-c7ccccc7)cc6)c5)cc4)c3c2c1 ; c1ccc(-c2ccc(N(c3ccc(-c4ccccc4)cc3)c3cccc(-c4ccc(-n5c6ccccc6c6sc7ccc(-c8ccccc8)cc7c65)cc4)c3)cc2)cc1 ; c1ccc(-c2ccc(N(c3ccc(-c4ccccc4)cc3)c3cccc(-c4ccc(-n5cc(Sc6ccccc6-c6ccccc6)c6ccccc65)cc4)c3)cc2)cc1 ; c1ccc(-c2ccc(N(c3ccc(-c4ccccc4)cc3)c3cccc(-c4ccc(-n5c6ccccc6c6sc7cc(-c8ccccc8)ccc7c65)cc4)c3)cc2)cc1] 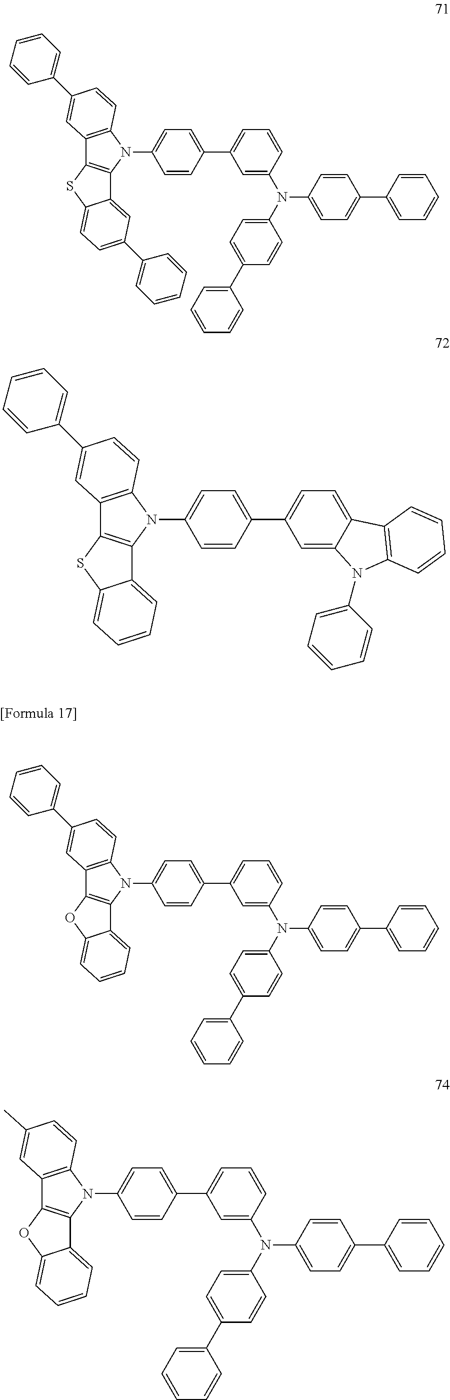 [SMILES: c1ccc(-c2ccc(N(c3ccc(-c4ccccc4)cc3)c3cccc(-c4ccc(-n5c6ccc(-c7ccccc7)cc6c6sc7ccc(-c8ccccc8)cc7c65)cc4)c3)cc2)cc1 ; c1ccc(-c2ccc3c(c2)c2sc4ccccc4c2n3-c2ccc(-c3ccc4c5ccccc5n(-c5ccccc5)c4c3)cc2)cc1 ; c1ccc(-c2ccc(N(c3ccc(-c4ccccc4)cc3)c3cccc(-c4ccc(-n5c6ccc(-c7ccccc7)cc6c6oc7ccccc7c65)cc4)c3)cc2)cc1 ; Cc1ccc2c(c1)c1oc3ccccc3c1n2-c1ccc(-c2cccc(N(c3ccc(-c4ccccc4)cc3)c3ccc(-c4ccccc4)cc3)c2)cc1] 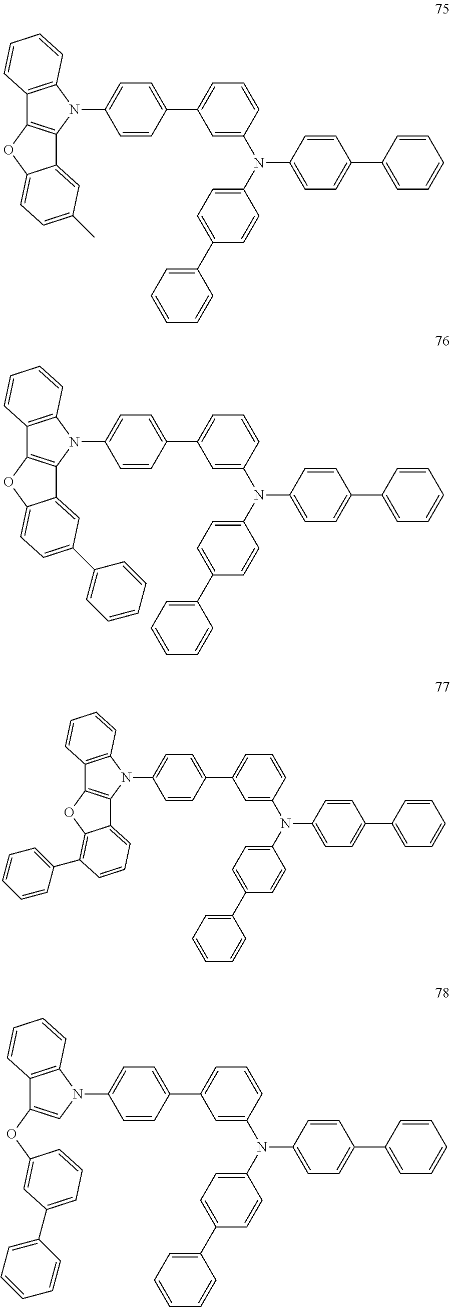 [SMILES: Cc1ccc2oc3c4ccccc4n(-c4ccc(-c5cccc(N(c6ccc(-c7ccccc7)cc6)c6ccc(-c7ccccc7)cc6)c5)cc4)c3c2c1 ; c1ccc(-c2ccc(N(c3ccc(-c4ccccc4)cc3)c3cccc(-c4ccc(-n5c6ccccc6c6oc7ccc(-c8ccccc8)cc7c65)cc4)c3)cc2)cc1 ; c1ccc(-c2ccc(N(c3ccc(-c4ccccc4)cc3)c3cccc(-c4ccc(-n5c6ccccc6c6oc7c(-c8ccccc8)cccc7c65)cc4)c3)cc2)cc1 ; c1ccc(-c2ccc(N(c3ccc(-c4ccccc4)cc3)c3cccc(-c4ccc(-n5cc(Oc6cccc(-c7ccccc7)c6)c6ccccc65)cc4)c3)cc2)cc1]
